(19) United States
(12) Patent Application Publication
(10) Pub. No.: US 2004/0236516 A1
(43) Pub. Date: Nov. 25, 2004

(54) Bioinformatics based system for assessing a condition of a performance animal by analysing nucleic acid expression

(76) Inventor: Richard Bruce Brandon, Kenmore (AU)

Correspondence Address: Heller Ehrman, White & McAuliffe, Suite 300, 1666 K Street NW, Washington, DC, 20006, US

(21) Appl. No.: 10/476,569

(22) PCT Filed: May 3, 2002
(86) PCT No.: PCT/AU02/00553

Related U.S. Application Data
(63) Continuation-in-part of application No. 09/896,941, filed on Jun. 29, 2001, now abandoned.

(30) Foreign Application Priority Data
May 4, 2001 (AU) PR 4809
May 4, 2001 (AU) PR4809

(51) Int. Cl. C12Q 1/68; G06F 19/00; G01N 33/48; G01N 33/50

(52) U.S. Cl. 702/20; 435/6

(57) ABSTRACT

A condition and ability of an animal to perform to its best ability may be determined by correlating gene expression with clinical and other data. The invention provides methods for assessing a performance animal's condition including the steps of collecting biological samples and clinical history, generating digital results on relative or absolute gene expression levels in the samples, transmitting the digital results via a communications network to a remote diagnostic server and associated database, comparing the results with information stored in the remote database and returning a report of the condition of the animal. A diagnostic system comprising a microarray, a microarray reader, a remote database for storing information from the reader, and a remote server receiving digital signals from the reader is also disclosed.

### FIELD OF THE INVENTION

The present invention relates to a bioinformatics-based method and system for appraisal, assessment and/or diagnosis of a condition of a performance animal and its capacity to perform to its best ability. In particular, the invention relates to a method and system comprising a centrally located database and data processor that respectively store and process information in relation to nucleic acid expression and a condition of a performance animal. The system is well suited for use with microarray and genechip technologies.

### BACKGROUND OF THE INVENTION

A condition of a performance animal, for example a racehorse, may typically be determined by conventional means such as a blood profile test and clinical appraisal. However, these tests are of limited value because a correlation between results of a blood profile test or clinical appraisal and a condition or state of a performance animal is minimal.

A blood profile test may be suitable for providing some information in relation to an animal that is clinically diseased or ill, but is rarely suitable for determining fitness to perform of an animal, particularly if the animal is healthy according to use of current clinical appraisal methods, and particularly if the animal cannot communicate information about its condition. Although blood profile tests are relatively inexpensive and easy to perform, they do not provide assessment of a wide range of conditions, correlations between test results and conditions of performance animals are poor, are limited to assessment of a few diseases, and are sometimes only useful in assessment of advanced stages of disease where clinical intervention is too late to prevent significant loss of performance.

Alternative diagnosis or assessment procedures are often complex, invasive, inconvenient, expensive, time consuming, may expose an animal to risk of injury from the procedure, and often require transport of the animal to a diagnostic centre.

A final report of the results of a blood test to an end user, eg. a trainer, often requires involvement of multiple parties each providing separate input to the report. For example, a veterinarian may collect a blood sample, the sample is transported or sent to a laboratory for analysis, personnel in the laboratory perform an analysis using machinery on the blood sample, automated results from the analysis, with or without a veterinary pathologist interpretation, are returned to the veterinarian who then interprets the results and provides a separate report to the trainer. The process is laborious, time consuming, subject to error and interpretation bias and may or may not contain information relevant to the end user.

Bioinformatics may be used with genetic based diagnosis of an animal's health. Bioinformatics is a rapidly growing discipline that combines biology and information technology. Bioinformatics is typically associated with genomic research projects, for example the “human genome project” which involves large-scale DNA nucleotide sequencing. Data in relation to nucleotide sequences, and annotated information in relation thereto, led to huge databases of information. Bioinformatics, has led to new database designs, methods for analysing nucleotide and amino acid sequence information, an ability to predict amino acid sequences and modelling nucleic acid and protein structures.

Bioinformatics has been used to study differential gene expression in tissues and cells, for example, differential expression between diseased and normal tissue. Often, Expressed Sequence Tags (ie. ESTs) from cDNA libraries are identified and sequenced for use as markers or tags for gene expression. An abundance of one or more ESTs in a cell may be determined and expression information stored in a database for comparison with known expression patterns for a condition of a tissue or cell.

One means for assessing a condition or health of an animal is performing a genetic assessment or genetic profile of the animal. Such an assessment may determine a condition of an animal based on expression or lack of expression of genes associated with a normal or abnormal phenotype. Accumulation of genetic information has rapidly grown -in light of new developments for genetic analysis, for example use of microarrays. Processing of such data has become complex and there is a need for a system not only for generating new genetic information, but also for processing the data so that useful information may be gained in an efficient manner which is easily accessible to end users.

Bioinformatics has been used to process genetic information that may result in diagnosis of an animal's state of health. As described in U.S. Pat. No. 6,287,254, phenotypic and genotypic data may be stored in a central database processing resource that is accessible to selected users. The genotypic data relates to DNA fingerprinting, genetic mapping, genetic background and genetic screening databases. Such genetic information is limited to congenital and heritable traits, thus changes in gene expression in response to factors such as diet and environment are not accounted for, nor are changes in the early stages of disease, nor are cases where gene penetrance is not complete. Also, genotypic data is compared with a limited panel of genetic markers for specific heritable traits that do not necessary relate to a changing condition of an animal in response to environmental, eg. non-genetic, factors. A health profile may be determined by statistically correlating phenotypic data with genotypic data. A report is generated that may be useful with an animal breeding program for selection and identification of suitable mating pairs.

U.S. Pat. No. 6,114,114 describes a method for comparing relative abundance of gene transcripts between healthy and diseased human tissue using high-throughput sequence-specific analysis of individual RNAs or their corresponding cDNAs. This provides a method and system for quantifying relative abundance of gene transcripts in a biological sample. A diagnostic test can be performed on an ill patient in whom a diagnosis has not been made. The patient's sample is collected, gene transcripts isolated and expanded to an extent necessary for gene identification and determination of the relative abundance of individual gene transcripts. Optionally, the gene transcripts are converted to cDNA and then the relative abundance determined. A sample of the gene transcripts are subjected to sequence-specific analysis and quantified.

These gene transcript sequences are compared against a reference database of the relative abundance of specific genes and their DNA sequences in diseased and healthy patients. The patient may be diagnosed as having a disease(s) with which the patient's data set most closely correlates. Because diseases are mostly species specific, due to variations in gene sequence between species, and due to variations between species in the relative abundance of different RNAs in tissues, the method described in U.S. Pat. No. 6,114,114 relates to gene expression in disease in the human. This U.S. patent describes identification of individual genes that are differentially expressed in abnormal and normal tissues. The patent does not provide a method for detecting or diagnosing a condition in a performance animal, or differentiating apparently normal animals, based on a pattern of gene expression or differences in gene expression.

Similarly, International application WO 01/25473 describes a method to assess the condition of a subject. This method includes the steps of: determining relative levels of RNA expression on a panel of genes using reverse-transcriptase polymerase chain reaction, retrieving relative RNA expression data from a remotely located database and comparing to the data with datasets and to a baseline. A user is provided with access to the remote database and information stored therein is transferred to the location of the user. In this manner, each user has access to the database and is thereby required to download and process the expression data at the user's location. Processing of the data may require bioinformatics skills and computer hardware and software to support data processing that may not be available to the user. Downloading large database files requires wide bandwidth and is time consuming, thus the described method may not be desirable for many users. The method is reliant on public knowledge of DNA sequences, public functional information on the selected genes for a panel, some prior knowledge of a disease or suspected disease so that a panel of appropriate genes is selected and downloading of appropriate data to a user's location. The method described does not use apparatus such as microarrays to determine absolute levels of RNA in a sample so that samples may be correlated without use of a baseline, or genes that have no a priori correlation to previously described disease or conditions. It does not appear that this method can be used to assess the condition of a performance animal without prior knowledge of species specific gene sequences, gene function, disease processes, prior knowledge of or suspected condition of an animal, and baseline sample data.

A method for a medical diagnostic advice system accessible via a computer network is described in U.S. Pat. No. 6,206,829. This method provides medical diagnosis of a condition based in part on a patient's history and patient provided description of symptoms. This method is not useful for conditions which require detailed physical examination and/or laboratory testing to provide a diagnosis, or where patient description of symptoms cannot be obtained. For example, this method is not suitable for diagnosing a condition that is not readily or physically detectable or communicable. In particular, this method would not be useful in diagnosing a condition in an otherwise healthy appearing individual, in a normal individual according to clinical appraisal and current diagnostic methods, or in an individual requiring differentiating information in relation to its level of performance, or in animals not capable of communicating information on a clinical history, or in diseased states that do not produce symptoms (carriers), or disease states that require specific laboratory tests for confirmation. This method also does not describe use of molecular biological methods, for example assessment of gene expression, in diagnosis.

The background art describes methods for diagnosing disease, or predisposition to disease using standard blood tests, which are limited to testing a few diseases and may have low sensitivity and specificity, and low correlation to a condition. These blood tests usually include a complete blood count, a differential count of white blood cells and measurement of serum electrolytes. More sensitive and specific blood tests are available based on the detection of antibodies or antigens or other metabolites but have the limitation that they are not generally used unless the animal is clinically ill or there are indications that such a test should be performed.

Invasive procedures are available for more accurate assessment for a broader range of diseases, however, such methods have inherent risks, and/or are costly and time consuming (for example, X-rays, scintigraphy, ultrasound, surgery and biopsy).

Genetic methods for diagnosing disease are often limited to specific genes that have already been identified which correlate with particular diseases. Genetic diagnostic methods may also be limited to human application because of dependence of such methods on information provided by the patient, information available in relation to a specific disease, or stage of disease, species and/or specific DNA sequence information, or datasets specific to a species.

The above mentioned background art does not describe a system for assessing or testing for a condition, level of performance, fitness to perform, response to or detection of drugs, response to vaccination, sub-classifying known disease, identification of new pathological descriptions of diseases or stages of diseases in a performance animal.

### SUMMARY OF THE INVENTION

The background art describes known methods for assessing expression of known genes. There is a need for a computer-based clinical support system capable of collecting and processing newly identified and known gene expression and clinical data, storing this data in a database, automatically or semi-automatically mining the data for assessment of a condition (including heuristic methods and rule-based methods), controlling the data stored within the database, and providing automated and useful interpretative information and patient specific reports to remote users.

The method and system of the invention uses molecular biological methods for determining nucleic acid expression, a communications network for transmitting data relating to nucleic acid expression for a performance animal, together with relevant clinical information and biochemical and haematological data, to a remote diagnostic server and associated database and central processor. The data is centrally processed by the diagnostic server at the remote database and compared to database contents, and a report of an animal's condition is generated at the central site and provided to the user at a remote location, for example a clinic. The data input into the database may also include an analysis by an expert biologist, geneticist, pathologist, veterinarian, bioinformaticist or the like. Accordingly, data sent by a user is processed using data stored in the central database, wherein the data has been analysed by experts and/or by a computer using rule-based instructions to thereby improve the accuracy and usefulness of the report. The method and system therefore provides a more informative report than may be obtained by the user performing an analysis by merely accessing a remote database of expression information. Further, the system of the invention provides a means for controlling access to valuable proprietary data stored within the database (ie. a user does not have direct access to the information of the database), less bandwidth is required sending less complex sample data compared to sending of large database files and processing is centrally located and thus more efficient.

The present invention provides one or more of the following: a clinically correlative, minimally invasive, sensitive and specific, convenient, accurate, rapid and relatively inexpensive system for providing assessment information for a condition, and ability of an animal to perform to its best ability. The invention is particularly useful in instances where there is no overt disease, or the animal is clinically healthy according to current methods, and the procedure is simply performed to gain further information about the capacity of a performance animal to perform to its best ability. Such a diagnostic method may be used to determine severity of a sub-clinical disease, its possible effect on performance, whether training should persist, level of risk associated with continued training and whether continued training may adversely affect future performance. Factors including subtle changes in diet, training regime, stable, or season may affect performance of an animal. It would be appreciated that in performance animals, being either human, horse, camel or dog, gene expression profiles or signatures relating to a particular condition in one species would be able to be used in other species, all being mammals and subject to similar conditions of performance. The method is therefore not reliant on known gene function in any particular performance animal species.

In one aspect the invention provides a method for assessing a condition of a performance animal including the steps of:

(a) determining in a sample obtained from a performance animal an abundance of an expressed target nucleic acid normalised to at least one reference nucleic acid and providing the normalised abundance of the target nucleic acid as a digital sample signal;

(b) transmitting via a communications network the digital sample signal of (a) to a remotely located diagnostic server and associated processor and database comprising digital information in relation to an abundance of the target nucleic acid which corresponds to a particular condition of the performance animal;

(c) processing the digital sample signal at the remotely located database to correlate the digital signal of step (a) with the digital information of step (b) thereby identifying a particular condition of the performance animal; and

(d) returning a report of the particular condition of the performance animal.

Preferably, the sample comprises at least one immune cell type.

More preferably, the at least one immune cell type is a white blood cell.

The normalised abundance of the target nucleic acid may be either a relative abundance or an absolute abundance.

Preferably, the normalised abundance of the target nucleic acid is an absolute abundance.

Preferably, the method further includes the step of determining in a sample obtained from the same performance animal in step (a), currently available routine biochemical and hematological parameters (blood profile test) and recording all available relevant clinical information in a standard format.

More preferably, the clinical information is transmitted via a communications network to the same remotely located diagnostic server and associated processor and database of step (b).

Preferably, the communications network is selected from the group consisting of: the Internet, an intranet, an extranet, wireless means or dedicated link (eg. ISDN).

In one form of the invention, the step of determining an absolute abundance of the target nucleic acid includes the steps of:

(i) detecting a first hybridised complex formed by at least one target nucleic acid and a perfect-complementary probe nucleic acid located on a solid support, thereby providing a digital perfect target signal;

(ii) detecting a second hybridised complex formed by at least one target nucleic acid having a same nucleotide sequence as the target nucleic acid of step (i) and a mismatch-complementary probe nucleic acid comprising a mismatched nucleotide in a central location of the mismatch-complementary probe nucleic acid when compared with a corresponding perfect-complementary probe, wherein the mismatch-complementary probe nucleic acid is located on a solid support and hybridisation thereto provides a digital mismatch or background target signal; and

(iii) comparing the digital perfect target signal of step (i) and the digital mismatch target signal of step (ii) to provide a digital signal of absolute abundance of the target nucleic acid.

Preferably, the respective hybridised complex of step (i) and step (ii) are detected by respectively labelling the target nucleic acids.

More preferably, the respective labelled target nucleic acid are labelled with biotin, Cy3 or Cy5.

Preferably, the respective labelled target nucleic acid is cRNA.

The solid support is preferably an array.

More preferably, the array is a microarray or similar device.

In another form of the invention, the step of determining a relative abundance of the target nucleic acid includes the steps of:

(A) detecting a hybridised complex formed by at least one sample target nucleic acid and a complementary probe nucleic acid immobilised on a solid support to provide a digital sample target signal;

(B) detecting a hybridised complex formed by at least one reference target nucleic acid comprising a nucleotide sequence different than the target nucleic acid of step (A), and a complementary probe nucleic acid immobilised on a solid support to provide a digital reference target signal; and

(C) comparing the digital sample target signal of step (A) and the digital reference target signal of step (B) to provide a digital signal of relative abundance of the sample target.

The reference nucleic acid may include any suitable nucleic acid characterised by a relatively constant level of expression.

The reference nucleic acid may be selected from the group consisting of: GAPDH, actin, and ribosomal 18S.

The respective complementary nucleic acids of step (A) and step (B) may comprise a perfectly complementary or homologous nucleotide sequence.

Preferably, the respective hybridised complexes of step (A) and step (B) are detected by respectively labelling the target and the sample target nucleic acid and reference target nucleic acid.

More preferably, the respective target and the reference nucleic acids are respectively labelled with Cy3, Cy5 or biotin.

The performance animal is preferably a mammal.

More preferably, the mammal is human, horse, dog or camel.

The performance of an animal may relate to its athletic ability and any condition that may enhance, hinder, impede or not change its expected ability.

The condition of the performance animal may comprise normal, apparently normal, pre-clinical disease, overt disease, progress and/or stage of disease, undiagnosed or unclassified conditions, presence of drugs, response to exercise, response to vaccines, therapies, nutritional states and response to environmental conditions.

The disease may comprise inflammation or involvement of the immune system; to include conditions affecting respiratory, musculoskeletal, urinary, gastrointestinal and adnexa, cardiovascular, reticuloendothelial, nervous, special senses, reproductive, and integument systems. Such examples in the horse include, laminitis, lameness, viral or bacterial disease, colic, gastritis, gastric ulcers, respiratory ailments, epistaxis, fractures, musculoskeletal damage or disorders and joint disease.

Another aspect of the invention relates to a diagnostic system comprising:

(I) an array comprising one or more probe nucleic acids immobilised to a surface, wherein the respective probe nucleic acids comprise nucleotide sequences hybridisable to a target nucleic acid;

(II) an array reader that detects hybridised complexes formed respectively by the target nucleic acid and the probe nucleic acid, whereby the array reader generates a digital signal of the respective detected hybridised complexes;

(III) a remotely located database storing information in relation to abundance of the target nucleic acid and clinical and blood profile data corresponding to particular conditions of performance animals;

(IV) a diagnostic server that receives the digital signal from step (I) and correlates the digital signal with information in the database to identify said particular condition and reports said particular condition; and

(V) a means for communicating between the array reader and the diagnostic server.

The probe nucleic acid may be a perfect-complementary nucleic acid comprising a nucleotide sequence perfectly complementary to the target nucleic acid, a mismatch-complementary nucleic acid comprising a mismatched nucleotide in a central location of the nucleic acid when compared with a corresponding perfect-complementary nucleic acid or a reference nucleic acid comprising a nucleotide sequence that is different than the target nucleic acid and hybridisable to a complementary reference target nucleic acid.

The array and array reader are remotely located from the central database and may be suitably located in a laboratory, veterinary clinic of other similar facility.

The diagnostic system may further comprise a means to display the report.

The present invention has advantages over current methods for diagnosing disease, for example laminitis (inflammation of the soft tissues in the hoof) in a racehorse. In many instances laminitis is sub-clinical, that is, the horse does not present clinically as lame. However, an owner or trainer may be concerned that the horse is not performing to the best of its ability. In this instance, a blood test and/or X-ray may traditionally be performed. However, subtle inflammation of the hoof will not be able to be detected by X-ray and will not be reflected in any abnormal values in current blood tests. Considerable expense through current test costs and lost training time, and inconvenience through transport of animals to diagnostic centres could be encountered with the risk of gaining little information on the exact condition or state of the animal, and whether and when it can perform to the best of its ability. Hence, the horse may have normal results from current tests, but actually have laminitis. Such a horse may not be performing to its best ability and the owner and trainer would remain oblivious to its condition. However, with use of the present invention, it may be possible to diagnose a horse having laminitis where other methods fail.

Another example of deficiencies of current blood tests is evident by methods for testing an athlete for use of illegal or prohibited performance-enhancing steroids. Current blood tests directly measure a level of a steroid in serum using equipment such as high performance liquid chromatographs, gas chromatographs or similarly sensitive equipment. These tests are not capable of detecting the steroid where the athlete is also using masking drugs, or where the athlete has not taken steroids for a period prior to the test being performed. The present invention may not directly detect a drug per se, but rather may detect an effect of a drug via detectable changes in nucleic acid expression. Such a change in nucleic acid expression may indicate presence of an otherwise undetectable drug in an athlete or performance animal.

It will be appreciated that the present invention may have one or more of the following advantages of being relatively inexpensive, accurate, convenient, rapid minimally invasive and sample results are processed at a central remotely accessible database and processor. Further, the present invention is not dependent on isolating a known gene of known function to determine a condition of an animal. The present invention may be used with a nucleic acid of known nucleotide sequence and expression level (gene transcript relative abundance) in a reference sample that is comparable with a nucleic acid expression level in a test sample. Although a preferred embodiment of the invention includes use of an array for determining an abundance of nucleic acid expression, other methods for determining nucleic acid expression are contemplated, including for example, Northern blot analysis, dot blotting, RT-PCR, RNAse protection, SAGE, differential expression and other methods for ascertaining gene expression that are known in the art.

### BRIEF DESCRIPTION OF THE FIGURES

FIG. 1 is a flow diagram illustrating dataflow steps as part of a computer system capable of delivery of remote diagnostic services.

FIG. 2 is a flow diagram showing steps for diagnosing a condition of an animal in accordance with the invention;

FIG. 3 is a diagram illustrating an environment for working the invention as shown in FIG. 2;

FIG. 4 is a flow diagram illustrating steps for preparing an array in accordance with an embodiment of the invention;

FIG. 5 is a flow diagram showing steps for determining a nucleic acid expression level in a biological sample; and

FIG. 6 is a flow diagram illustrating steps for building a database in accordance with an embodiment of the invention.

### DESCRIPTION OF PREFERRED EMBODIMENTS

Definitions

Unless defined otherwise, all technical and scientific terms used herein have the meaning as commonly understood by those of ordinary skill in the art to which the invention belongs. Although any methods and materials similar or equivalent to those described herein can be used in the practice or testing of the present invention, preferred methods and materials are described. For the purpose of the present invention, the following terms are defined below.

The term “bioinformatics” refers to a discipline of using computers to collate and form datasets of interest to biologists. Usually the term is used to refer to databases of nucleotide and amino acid sequences, and of mutations, disease and gene functions.

The term “nucleic acid” as used herein designates single or double stranded total RNA, mRNA, RNA, cRNA and DNA, said DNA inclusive of cDNA and genomic DNA.

The term nucleic acid also comprises modifications, for example, chemical base substitutions and nucleic acid comprising a polyamide backbone such as peptide nucleic acids (PNAs) as described in International Patent WO 92/20702 and (Egholm, et al., 1993, Nature, 365, 560) herein incorporated by reference. It will also be appreciated that the backbone of a nucleic acid may comprise a peptide-like unit as well as a unit of sugar groups linked by phosphodiester bridges, optionally substituted with other groups such as phosphorothioates or methylphosphonates.

The term “isolated nucleic acid” as used herein refers to a nucleic acid subjected to in vitro manipulation into a form not normally found in nature. Isolated nucleic acid includes both native and recombinant (non-native) nucleic acids.

The term “target nucleic acid” means a nucleic acid that has been labelled. A target nucleic acid may be a single or double-stranded oligonucleotide or polynucleotide, suitably labelled for the purpose of detecting a complementary nucleotide sequence of a probe nucleic acid that may, for example, be attached to a solid support, for example a microarray. Useful labels include, for example, biotin, Cy3 and Cy5. A single stranded probe may be synthesised from cDNA thereby making antisense RNA or sense RNA. The target nucleic acid may be labelled using any means including for example, radioactive and non-radioactive labels. In one embodiment of the invention, a labelled target is a labelled cRNA. The labelled cRNA is synthesized from double stranded cDNA using a DNA dependent RNA polymerase. The cDNA may be synthesised from mRNA isolated from a sample using methods well known in the art for making cDNA libraries. The labelled cRNA thus corresponds to an amount of mRNA, or expressed nucleic acid, in a sample.

The term “probe” used herein refers to a nucleic acid that has been immobilised. For example, a probe may include a nucleic acid immobilised to a microchip, membrane, well, dish or any other suitable surface.

An “oligonucleotide” has less than eighty (80) contiguous nucleotides, whereas a upolynucleotide is a nucleic acid having eighty (80) or more contiguous nucleotides. An oligonucleotide may be used for example as a probe, primer or attached to a substrate as an array element or built onto an array.

A “primer” is usually a single-stranded oligonucleotide, preferably having 20-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. The invention in one embodiment uses oligo-dT primers which may anneal to a polyA region of mRNA. In another embodiment, gene-specific primers may be used which anneal to complementary isolated nucleic acid from a biological sample, to amplify nucleotides therebetween. Use of these primers is provided in more detail hereinafter.

Nucleic Acid Sequence Comparison

Terms used herein to describe sequence relationships between respective nucleic acids include “comparison window”, “sequence identity”, “percentage of sequence identity” and “substantial identity”. Optimal alignment of sequences for aligning a comparison window may be conducted by computerised implementations of algorithms (for example ECLUSTALW and BESTFIT provided by WebAngis GCG, 2D Angis, GCG and GeneDoc programs, incorporated herein by reference) or by inspection and the best alignment (i.e., resulting in the highest percentage homology over the comparison window) generated by any of the various methods selected.

Reference may be made to the BLAST family of programs as for example disclosed by Altschul et al., 1997, Nucl. Acids Res. 25 3389, which is incorporated herein by reference. A detailed discussion of sequence analysis can also be found in Chapter 19.3 of CURRENT PROTOCOLS IN MOLECULAR BIOLOGY Eds. Ausubel et al., (John Wiley & Sons, Inc. 1995-1999).

The term “sequence identity” is used herein in its broadest sense to include the number of exact nucleotide matches having regard to an appropriate alignment using a standard algorithm, having regard to the extent that sequences are identical over a window of comparison. “Sequence identity” may be understood to mean the “match percentage” calculated by the DNASIS computer program (Version 2.5 for windows; available from Hitachi Software engineering Co., Ltd., South San Francisco, Calif., USA).

As generally used herein, a “homolog” shares a definable nucleotide sequence relationship with a nucleic acid.

In one embodiment, nucleic acid homologs share at least 60%, preferably at least 70%, more preferably at least 80%, and even more preferably at least 90% sequence identity with the nucleic acids of the invention.

In yet another embodiment, nucleic acid homologs hybridise to nucleic acids under at least low stringency conditions, preferably under at least medium stringency conditions and more preferably under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

In DNA, complementary bases are:

(i) A and T; and

(ii) C and G.

In RNA, complementary bases are:

(i) A and U; and

(ii) C and G.

In RNA-DNA hybrids, complementary bases are:

(i) A and U;

(ii) A and T; and

(iii) G and C.

Modified purines (for example, inosine, methylinosine and methyladenosine) and modified pyrimidines (thiouridine and methylcytosine) may also engage in base pairing. Hybridise and hybridisation may also refer to pairing between complimentary modified nucleic acids for example PNA and DNA, and PNA and RNA respectively.

A labelled target nucleic acid and complementary probe nucleic acid located on an array may hybridise with each other. A “prefect-complementary” probe nucleic acid comprises a nucleotide sequence that is exactly matched with a complementary target nucleic acid. A “mismatched-complementary” probe comprises a mismatched nucleotide when compared with a prefect-complementary probe. Preferably, the mismatch is in a central location of the nucleic acid.

“Stringency” as used herein, refers to temperature and ionic strength conditions, and presence or absence of certain organic solvents and/or detergents during hybridisation. The higher the stringency, the higher will be the required level of complementarity between hybridising nucleotide sequences.

“Stringent conditions” designates those conditions under which only nucleic acid having a high frequency (percentage) of complementary bases will hybridise.

Stringent conditions are well known in the art, such as described in Chapters 2.9 and 2.10 of Ausubel et al., supra, which are herein incorporated by reference. A skilled addressee will also recognise that various factors can be manipulated to optimise the specificity of the hybridisation. Optimisation of the stringency of the final washes can serve to ensure a high degree of hybridisation.

As used herein, an “amplification product” refers to a nucleic acid product generated by nucleic acid amplification techniques.

Suitable nucleic acid amplification techniques are well known to the skilled addressee, and include PCR as for example described in Chapter 15 of Ausubel et al. supra, which is incorporated herein by reference; strand displacement amplification (SDA) as for example described in U.S. Pat. No 5,422,252 which is incorporated herein by reference; rolling circle replication (RCR) as for example described in Liu et al., 1996, J. Am. Chem. Soc. 118 1587 and International application WO 92/01813; International Application WO 97/19193, which are incorporated herein by reference; nucleic acid sequence-based amplification (NASBA) as for example described by Sooknanan et al.,1994, Biotechniques 17 1077, which is incorporated herein by reference; ligase chain reaction (LCR) as for example described in International Application WO89/09385 which is incorporated herein by reference; and Q-β replicase amplification as for example described by Tyagi et al., 1996, Proc. Natl. Acad. Sci. USA 93 5395 which is incorporated herein by reference. Preferably, amplification is by PCR using primers and nucleic acids as described herein.

The term “array” refers to an ordered arrangement of hybridisable array elements. The array elements are arranged so that there are preferably multiple copies of a single element as an internal control, enough copies of positive and negative controls to determine background hybridisation. For example Affymetrix uses a “perfect match” (ie. perfect-complementary nucleic acid) and “mismatch” (ie. mismatch-complementary nucleic acid) method to measure this parameter. A suitable number of copies of the single element are required to specifically and sensitively hybridise to its complementary nucleic acid (or near complementary for mismatch nucleic acids). One or more different array elements may be immobilised to a substrate surface. Preferably at least 10 array elements, more preferably at least 100 array elements, and even more preferably at least 5,000 array elements are immobilised to a substrate surface. Where an array surface is small, for example 1 cm², the array may be referred to as a “microarray”. Furthermore, hybridisation signal from respective array elements is individually distinguishable. In one embodiment, an array element comprises a polynucleotide sequence. In another embodiment, an array element comprises an oligonucleotide sequence.

“Element” or “array element” in an array context, refers to a hybridisable nucleic acid arranged on a surface of a substrate, including microspheres.

“Biological sample” is used in its broadest sense and may comprise a tissue, for example from a biopsy; bodily fluid, for example blood, sputum, urine, bronchial or nasal lavages, joint fluid, peritoneal fluid, thoracic fluid; a cell; an extract from a cell, for example, an organelle or nucleic acid inclusive of a chromosome, genomic DNA, RNA (total and mRNA), and cDNA.

A “blood profile test” is defined herein as use of current technology to assess blood of an animal, and may include cell counts, cell appraisal and other biochemical, immunological and cellular tests.

“Clinical appraisal” is defined herein as use of observation, experience and/or use of more sophisticated diagnostic techniques. Alternative diagnostic techniques used to gain more information on conditions of performance animals include tests on lavages taken from body cavities, urine tests, bronchoscopy, ultrasound, MRI, CAT scans, X-rays, scintigraphy, and investigative surgery and tissue biopsy.

A “condition or state of an animal” refers to any influence, external or internal, that may hinder, enhance or not change the capacity of an animal to perform to its best ability.

The term “up-regulated” refers to mRNA levels encoding a gene which are detectably increased in a biological sample from a test animal compared with mRNA levels encoding the same gene in a biological sample from normal animal.

The term “down-regulated” refers to mRNA levels encoding a gene which are detectably decreased in a biological sample from a test animal compared with the mRNA levels encoding the same gene in a biological sample from normal animal.

The term “normaf” is used herein to refer to an animal which does not have any visible abnormalities or known performance hindrance or enhancement, as detected by an assessment by for example, a trainer, owner(s), own person, veterinarian, practitioner, independent authorities or bodies or through the use of for example a clinical appraisal, routine blood profiles, current available diagnostic technologies.

The present invention has applications including, for example, in instances where there is no overt disease, or the animal is healthy, and the procedure is performed to gain further information about a capacity of a performance animal to perform to its best ability. Such a diagnostic method may be used to determine severity of a sub-clinical disease, its possible effect on performance, whether training should persist, level of risk associated with continued training and whether continued training may adversely affect future performance. Factors including subtle changes in diet, training regime, stable, or season may affect performance of an animal.

Current Methods for Diagnosis of a Disease

Diagnosing a disease or determining risk of a disease using present genetic tests has limitations. For example, a cause of combined immunodeficiency disease (CID) in Arabian horses is known to be genetically based. As described in U.S. Pat. No. 5,976,803 an abnormal copy of the gene can be detected in DNA isolated from the animal using a DNA-based diagnostic test such as polymerase chain reaction (PCR). The gene responsible for CID and an exact DNA sequence of the normal and abnormal genes are conveniently known. However, in many instances conditions and disease are affected by and caused by variations within one gene, unknown genes, or through contributions from many genes. In many instances, the only evidence that a gene or group of genes may be responsible for altered conditions and disease in animals is through correlative statistical data between variations in non-protein coding DNA (intergenic regions or microsatellites) and clinical observations. Genes may also be suspected of causing a condition, but not yet proven, or the gene may be known but an exact nucleotide sequence or abnormality in the gene causing a condition is not known. Accordingly, genetic testing limited to only known genes that cause a particular disease are of limited value.

Microarrays Currently Used in Disease Diagnostics

Other current genetic tests include determining levels of gene expression in cells using microarrays, or other devices or methods capable of measuring levels of gene expression. The use of gene expression tests to compare cell populations is well known in the art. Such tests have been used to diagnose a disease state by measuring specific mRNA levels in peripheral blood leukocytes described in U.S. Pat. No. 6,190,857, incorporated herein by reference. In particular detecting the levels of mRNA for the genes IL8 or ILI0 in diseased state compared to normal state to determine presence of prostate cancer in humans.

Another example of such tests has been used to determine specific genes that are differentially expressed in normal and diseased tissue in humans. This has been used to assess a condition of a patient and is described in U.S. Pat. No. 6,194,158 that relates to gene expression in relation to brain cancers such as glioblastoma. A nucleic acid identified in such a manner and described in this patent may encode a complete or partial gene of interest, which may be attached to a substrate, for example a microarray, to assess relative gene expression of the differentially expressed gene.

A further extension of the use of gene expression technology has been used in diagnosis (class prediction), sub-classification (class discovery) and subsequent choice of therapy of leukemic cancer in human (Golub, 1999, Science 286 531), herein incorporated by reference. A further extension of the use of relative gene expression technology has been used to predict the clinical outcome of breast cancers and to determine a treatment regime in human breast cancer (Khan, 2001, Nature Medicine 7 673). Another extension of the use of gene expression technology in monitoring disease state and response to therapies has been described in U.S. Pat. Nos. 6,218,122, and 6,203,987 where an expression value for a gene-set is used as a basis for comparison between diseased and normal cells. Diagnosis and sub-classification of disease and disease prognosis is possible in these examples because a limited number of genes are differentially expressed, the condition is well defined, current tests can be used to diagnose and classify the disease, or stage of disease and/or symptoms are clinically obvious, or there are other methods of co-determining the clinical course of a disease.

In contrast with the above, determining a condition of a performance animal when there is no specific data on previous treatments or conditions relies on detection of differential expression of a large number of genes and correlation to previous data collected from a large number of samples where the clinical condition of the animals has been well documented and is not necessarily either clinically obvious, or current tests show no definitive diagnosis or classification of disease.

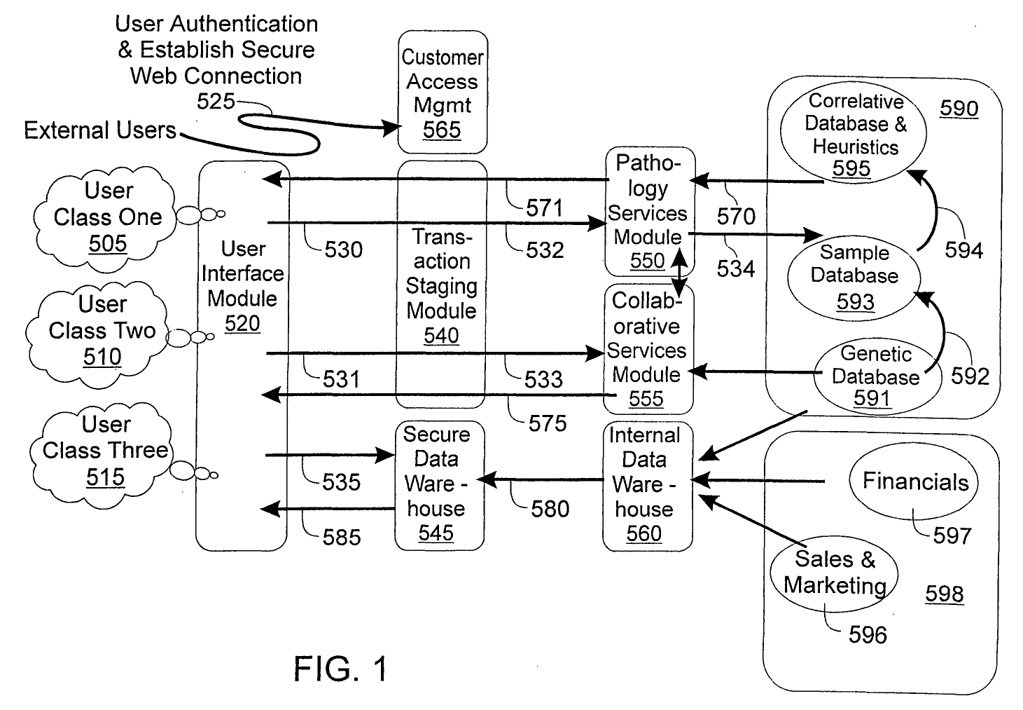

FIG. 1 is a flow diagram illustrating one embodiment of information technology architecture and data flow as part of a remote delivery service process of the invention. External users are shown as Class One 505, Class Two 510, and Class Three 515 that are interested in obtaining information regarding their respective gene expression results when using the proprietary gene expression analysis service. These users may include, for example, pathology laboratories, drug laboratories, pharmaceutical companies, collaborators, medical and/or veterinary practitioners or similar, owners of performance animals, athletes and/or athletic trainers. Each of these users 505, 510, 515 will be interested in different aspects of the gene expression results and will therefore interact in a different fashion, but all will interact remotely via an user interface module 520.

Interface 520 may, for example, be a browser-based interface as found on most computers and delivered via web pages on the world-wide-web (the Internet). The initial interaction to the user interface module 520 will be via a controlled firewall and web server. The firewall will be the first line of defence against unwanted and unauthorised intrusion. Port blocking techniques and protocol restrictions will be imposed at the firewall. The firewall and web server environment will be fully maintained with the latest security patches to ensure currency of protection against hackers and intrusion. Each user will establish a secure connection 525 (user authentication and establish secure web connection) to ensure confidential identification in both directions for the user and service delivery provider. The security is managed by a customer access management system 565 that controls access of users 505, 510, 515. Such security measures are commonly used in the art and one embodiment would be use of SSL (secure socket layer) technology and digital signatures. Further security layers can be added at this interface if required and might include challenge/response component such as continuously changing numerical keys in possession of the user and available in plastic card format and trusted networks.

Class One and Two Users 505, 510 are shown sending information as a query 530 and 531, that includes a question regarding health or condition status of an animal (interpretation request), sample details, gene expression results, clinical information, pathology laboratory results, gene identities, gene sequences, collaborative requests, etc. Class Three Users 515 are shown sending information 535 as a query including interrogation requests regarding a health status of individual animals/athletes or groups of individual animals/athletes.

Queries 530 and 531 may contain formatted gene expression and clinical information as a request, one such embodiment would employ the use of digitally signed XML documents to ensure authenticity and content of the request. Other authentication, authorization and encryption and key management standards will be applied as they become available.

As a further security measure to protect central databases 590, from outside unauthorised access, queries are temporarily stored in a transaction staging module 540 and queries 532 and 533 will be drawn into respective pathology service module 550 and collaborative services modules 555 only on request from the service module. This process may employ a second firewall and may be configured to further restrict network traffic. This firewall will only permit internal requests from 550 555 560 to pass through the firewall. All other network traffic will be blocked as will unnecessary ports and protocols. Respective pathology services module 550 and collaborative services module 555 include special software capable of servicing requirements of the different types of users 505, 510. Pathology services module 550 and collaborative services module 555 are shown in communication with each other. Core central databases 590 store genetic information (genetic database) 591, sample and gene expression information (sample database) 593, and correlative data (correlative database & heuristics) 595. The genetic information stored in genetic database 591 is used to create gene expression devices Design details 592are also stored in the sample database which contains gene location information on the device and are used to interpret results from such a device.

The genetic database 591 is also used to provide gene identification and gene sequence information to collaborative services module 555 and collaborative services 575 (eg. interpretations, gene lists and gene sequences) to Class Two users 510. Information in the sample database 593 can be clustered together based on similarity using computer algorithms such as K-means, principal component analysis (PCA) and self-organising maps, commonly available in packages provided by companies such as spotfire, silicon genetics, and at higher levels of interpretation, Omniviz. These clusters amount to identified correlations 594 between gene expression and sample information and are stored in various formats, in the correlative database 595. An heuristic o [symbol] eural network or rule-based computer software system pre-programmed with rules or training sets takes queries 534 (eg. expression details and sample details), stores these details in the sample database 593 and then compares the query pattern to those already stored in the correlative database 595 and produces standardized reports and correlation details 570 (according to the rules of the heuristic program). Correlation details are converted to useful information such as gene expression correlation results, for example a fully formatted report to include interpretations 571 and interpretations 575 (and optionally genes lists and gene sequences) and are securely delivered back to the requestor via the internet to Class One and Two users 505, 510.

Financials database 597 keeps track of details including for example accounting, purchasing and payroll details. Sales and marketing database 596 keeps track of items such as sales and marketing details, client details, customer relations management and stock management. Internal data warehouse 560 receives information from databases 590, 596 and 597. This internal data warehouse 560 will only be accessed by authorized internal users conducting legitimate business activities. A secure (internal) data warehouse 545 services the needs of Class Three users 515. Specific (and confidential) information 580 is extracted from internal data warehouse 560 that is then stored in secure customer data warehouse 545 where authorized users 515 can query 535 (for example as interrogation requests), specific and confidential information such as clinical history information, pathology results and interpretations. This information is presented in a secure user-friendly and/or visual format 585 in relation to individuals or groups of athletes or performance animals, and/or time series of results.

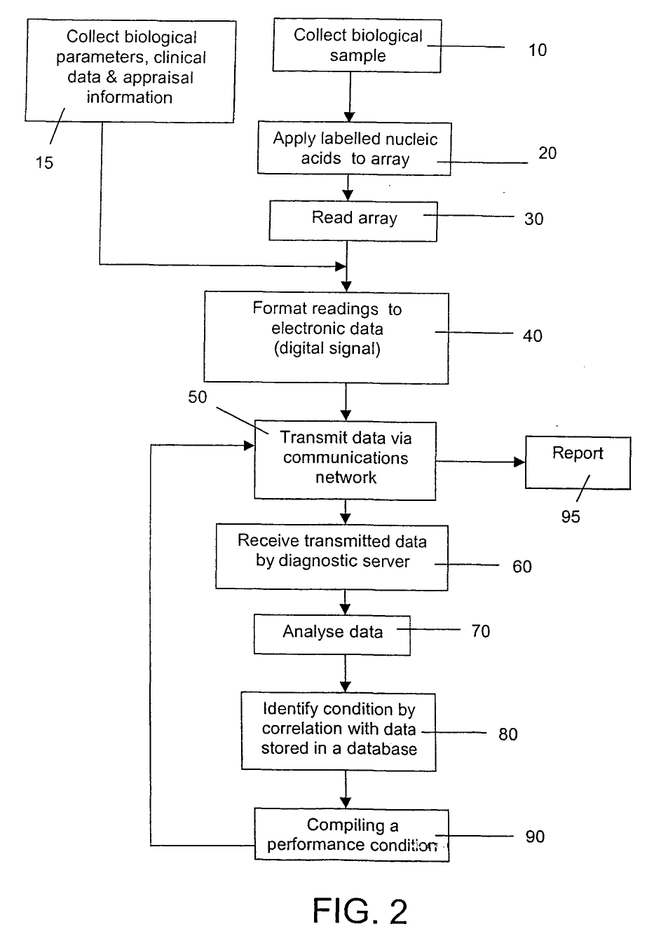

FIG. 2 is a flow diagram of one embodiment of the invention showing steps for assessing a biological sample for diagnosing or assessing a condition of an animal. A user collects a biological sample 10, for example a blood sample from a horse. At the same time, biological parameters including biochemical and haematological parameters, clinical data (including blood profile tests) and appraisal information are collected and recorded in a standard format 15, for example by filling in a standard form. The biological sample 10 is processed so that nucleic acids contained therein are detectable when hybridised with a complementary (or mismatch-complementary) nucleic acid located on an array 20. The nucleic acid may be detectable by a label incorporated therein, for example a target nucleic acid. Preferably, the array 20 is a device such as a microarray which is read 30 by standard methods and equipment common to the art to identify and measure relative abundance or absolute abundance of those nucleic acids from the biological sample which have bound to probe nucleic acids immobilised as part of array 20 (inclusion of a reference sample run in parallel allows for the calculation of the relative abundance of target nucleic acids, whereas a method developed by the company Affymetrix, Inc (the “Affymetrix system”) as described at their website “affymetrix.com” relies on internal references).

Array 20 may comprise a large number of probe nucleic acids, eg. 1000's of nucleic acids. A large number of probe nucleic acids may be particularly useful if an animal is not presenting with any visible signs of poor condition, eg. overt disease. Accordingly, in one embodiment, labelled target nucleic acids of a sample are first applied to an array comprising a “full-screen” of target nucleic acids (eg. 1,000's of nucleic acid probes that represent most or many of the nucleic acids expressed in a sample). Based on results from the full-screening, the labelled nucleic acid targets may be applied to a sub-set of the full-screen, eg. a selected panel of nucleic acid targets that may be associated with a particular condition, for example, respiratory diseases, drug consumption, etc.

Data from the read microarray 30 and clinical data and appraisal information 15 is formatted 40 and transmitted via a communications network 50, for example the Internet, to a remote diagnostic server 60. It will be appreciated that transmission of the formatted data to the remote diagnostic server 60 requires less bandwidth than transmitting database information to the user and less skill and time on behalf of the user. The transmitted data is analysed 70, for example by comparison to a database of previously collected information in relation to clinical information and expression levels (relative abundance) of the nucleic acids applied to the microarray 20. Also, experts, for example, bioinformaticists, biologists, doctors, pathologists, and the like may analyse the data to provide additional useful information. The analysis enables correlation to a condition 80. In this manner, the expression levels (relative or absolute abundance) of the nucleic acid probes applied to the microarray 20 are correlated with previously collected data relating to known conditions stored in a database 80 and compiled 90. The database may also store information in relation to an identity of known nucleic acids, nucleotide sequence on the array and/or location of nucleic acids on the array, its biological function and links to other databases.

Results in relation to health and performance condition are transmitted via a communications network 50 and may also be provided to the user as a report 95, for example a hardcopy printout or visually on a computer monitor.

The described system has advantages of requiring low bandwidth for transmitting sample data and final report between user and remote database/processor, data processing is centralised and more efficient, expert analysis of the sample data is centralised, the computer software may incorporate heuristic methods thereby minimising human interaction, the possibility of user and interpretation bias is avoided, and information stored in the commercially valuable database is under strict control and does not require direct access by an outside user. The steps are described in more detail hereinafter.

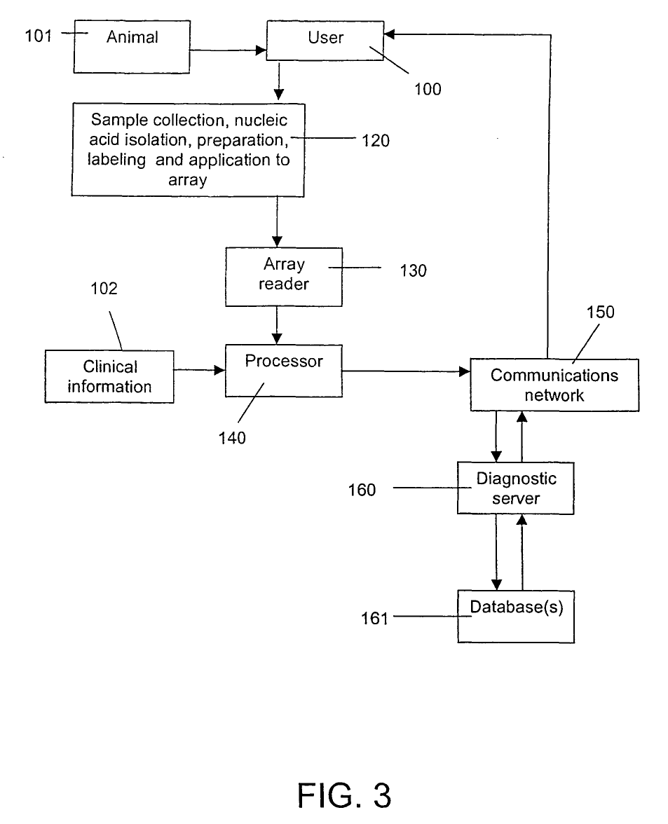

FIG. 3 shows an environment for working the method described in FIG. 2. A user 100, which may be a veterinarian or practitioner, collects a sample 120 from an animal 101, for example a blood sample from a horse or athlete. Concurrently, information in relation to a condition of the animal is collected in a standard format 102. The sample is collected, nucleic acids isolated therefrom, prepared and applied to an array 120 and the array is read by an array reader 130. Data from the array reader 130 and clinical appraisal and condition information 102 is entered into a computer and formatted by a processor 140, which may be for example, a laptop computer with a modem. The formatted data is transmitted via a communications network 150, for example the Internet. A remote diagnostic server 160 receives the transmitted data and the data is compared with a database(s) 161 which stores data, for example, data in relation to nucleic acid location on an array, expression level (relative abundance or absolute abundance) of a nucleic acid hybridised with a corresponding nucleic acid on an array, and data correlating nucleic acid expression level and performance, health, or condition of an animal.

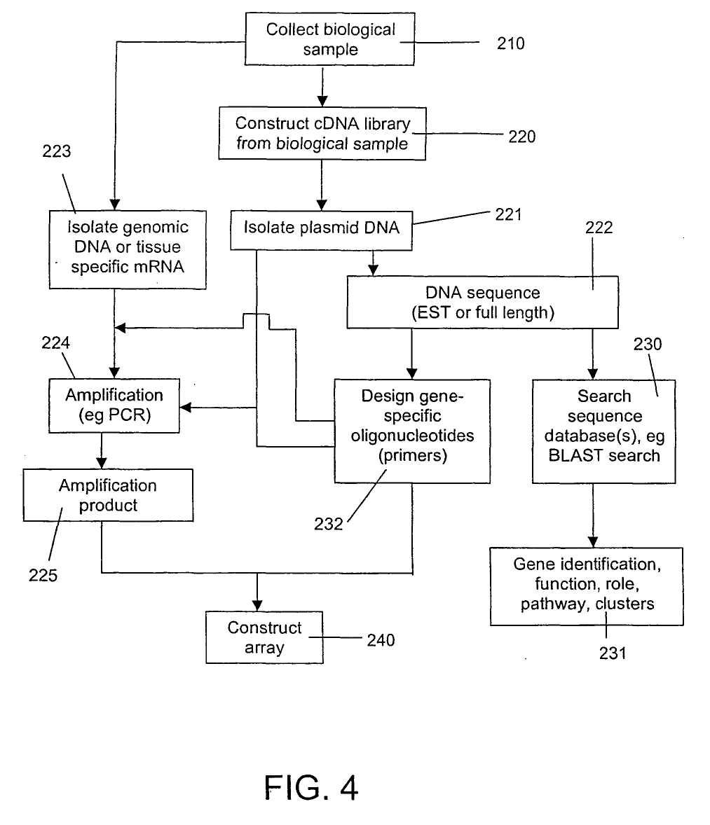

FIG. 4 is a flow diagram illustrating steps for preparing an array in accordance with the invention. A biological sample 210 is collected from an animal. Biological sample 210 may comprise for example, a blood sample (preferably white blood cells isolated therefrom), urine sample or tissue sample (including fetal tissues and tissues in various stages of development). A specific aim of collecting the biological sample is to isolate and sequence as many relevant genes from the sample for use on an array. Thousands of nucleic acids may be isolated that may form a large number of probes for a broad screening of an animal's genetic make-up or gene expression pattern.

Nucleic acids are isolated from the biological sample. In one instance the sample may be used to prepare genomic DNA or tissue specific mRNA 223. In another instance RNA is isolated from the biological sample 210 and a cDNA library 220 is prepared from the isolated RNA. Plasmids 221 comprising cDNA inserts from library 220 may be sequenced 222 from either or both 5′ and/or 3′ end of the nucleic acid. Preferably, sequencing is from the 3′ end. Sequences may comprise Expressed Sequence Tags (EST). If an isolated nucleic acid does not encode a full-length gene (eg. an EST), a partial nucleic acid may be used as a probe to isolate a full-length nucleic acid. Alternatively, or in addition, EST sequence information may be compared directly with a sequence database 230, for example GenBank, and a search for related or identical sequences performed. Putative gene identification and function 231 may be determined from a search, for example a BLAST search performed in step 230. By determining the number of times each gene is represented in the library, a computer may be programmed to enable the normalisation and standardisation of the relative abundance data of mRNAs in a sample.

Gene-specific oligonucleotides 232 may be synthesised using information from EST or full-nucleotide sequence 222 data. Gene-specific oligonucleotides 232 may be used as amplification primers to amplify (step 224) a region of a corresponding nucleic acid. The nucleic acid used as template to amplify a region of corresponding nucleic acid may be, for example, isolated plasmid DNA 221 and/or genomic DNA, cDNA or mRNA (eg. used with RT-PCR) 223. The nucleic acid thus prepared can be used directly as the nucleic acids for attaching to an array 240. Amplification products 225 may also be generated using non-gene-specific primers (eg. oligo-dt, plasmid sequence flanking a nucleic acid of interest). Oligonucleotides corresponding to a gene 232 may also be used on array 240, alternatively the oligonucleotide corresponding to known sequence can be built successively nucleotide by nucleotide on a support using Affymetrix methodology such as that in U.S. Pat. No. 5,831,070, incorporated herein by reference.

In one embodiment, the step relating to constructing cDNA 220 and isolating plasmids 221 comprising the cDNA may be omitted. In this embodiment, isolated genomic DNA or tissue specific mRNA 223 is used as a template to make amplification product 225 by amplification using gene-specific primers 232. Amplification product 225 may be attached to array 240.

Nucleic acids attached to or built onto array 240 preferably represent most, more preferably all, expressed genes in a given tissue from an animal of interest. For example, for a complete diagnostic test for racehorse blood, the array should contain genes expressed in the cells of blood under various conditions and at various stages of cell differentiation.

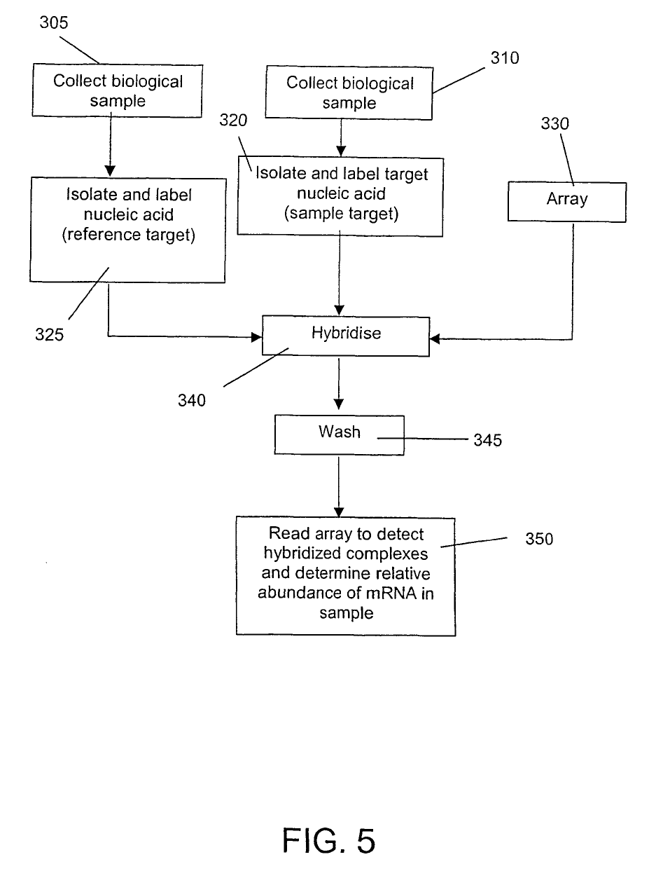

FIG. 5 shows a flow diagram comprising steps for determining gene expression in biological samples comprising both reference target 305 and sample target 310. Nucleic acids, in particular RNA (total RNA or mRNA), are isolated from biological samples 305 and 310, which may be the same sample. cDNA is prepared from the RNA and the cDNA is labelled resulting in labelled targets 320 and 325. Alternatively, or in addition, CDNA may be used as a template to synthesise labelled antisense RNA for use as targets 320 and 325. Reference target 325 may be provided as a previously prepared labelled target of known concentration. Accordingly, reference target 325 need not be synthesised in parallel with each sample target. Internal controls for reference target 325 and sample target 320 provide a means for normalising and scaling relative probe concentrations.

Sample target 320 and reference target 325 are hybridised with array 330 in step 340. Array 330 may, for example, have been prepared by steps shown in FIG. 4. The hybridised array is washed 345 to remove non-specific hybridisation of targets 320 and 325. It will be appreciated that one skilled in the art could select different stringency conditions of wash 345 as required. Array 330 is read in an array reader 350 to determine relative abundance of RNA in the original sample, which correlates with expression of the corresponding gene in the biological sample.

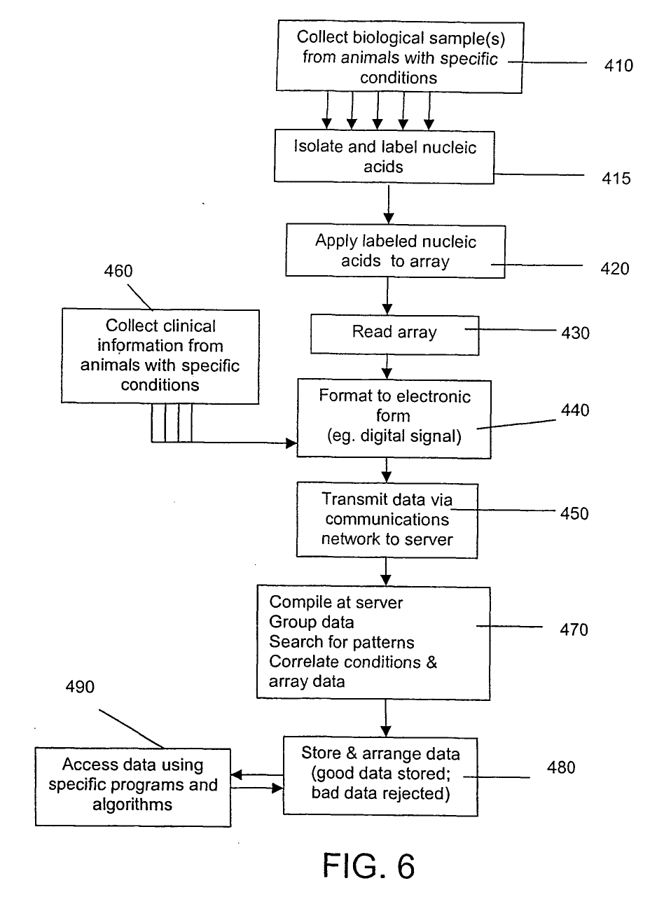

FIG. 6 is a flow diagram illustrating steps for building a database in accordance with the invention. Biological samples 410 are collected from animals having specific known condition(s). Preferably, a statistically relevant number of biological samples 410 are collected from a variety of normal animals to establish a normal reference range of nucleic acid abundance levels. This should account for natural variation, including that associated with state of fitness, sex, age, season, breed and diurnal changes. Nucleic acids are isolated and labelled 415 from sample 410, thereby forming respective target nucleic acids. The labelled target nucleic acids 415 are applied to array 420, which may be prepared as described in FIG. 4. The array is read 430 and data formatted 440 into an electronic form, for example a digital signal, suitable for transmission via a communications network 450. Clinical information from clinical appraisal, in relation to conditions of animals of interest is measured, documented and compiled 460. The clinical information is preferably collected in a standard format, and for example, variable states such as the level of fitness or body score (fatness) may be assigned given a value or number (for example between 1-10). Specific clinical conditions may be graded (for example between 1-10) and assigned a unique and standard identifier. An example of such a system is currently used in clinical medicine and veterinary science and termed SNOMED or SNOVET (Standardised Nomenclature of Medicine or Veterinary Science), where a clinical condition can be described using a numerical system. This system has not been used for describing the normal condition or the ability of a performance animal to perform to its best. A numerical grading system could also be used to standardise the collection of such data, for example, time spent on a treadmill is a strong indicator of exercise tolerance, as is blood concentration of oxygen and ability to transport oxygen. Conditions may include disease, response to drugs, training, nutrition and environment. The clinical information 460 is formatted into electronic form 440, for example a digital signal, suitable for transmission via a communications network 450.

The process is repeated such that a collection of several array readouts for particular conditions are made. A standard range (for example, a population median of 95%) of values for each of the represented genes and its relative abundance can be calculated. This reference range can then be used as a comparison to test sample results.

Nucleic acid expression information from a read array 430 for a target sample is correlated with previously measured conditions 460 to provide information on nucleic acid expression level (abundance or relative abundance) with any previously measured condition. This information is compiled at server 470 and good data is stored and bad data rejected 480. The compilation process includes collection of a large enough set of array readout information for a particular condition so that inferences can be drawn on gene expression profiles and conditions. The compilation 470 may also include use of sophisticated pattern recognition and organisational software and algorithms (examples common to the art include algorithms such as K means, Nova and Mann Whitney, Self Organising Maps, principal component analysis, hierarchical clustering—any one of which is available as part of proprietary software packages) such that expression patterns that differ to normal or expected condition can be identified. Concurrently, comprehensive clinical information 460 for animals may be collected and biological samples 410 tested on arrays so that correlations can be made between any clinical observation and array data. In this manner a database is created comprising data on nucleic acid expression which may include data correlating any desired condition, for example normal and specific abnormal condition(s), with nucleic acid expression. The stored data 480 may be accessed using specific programs and algorithms 490.

Throughout this specification, unless the context requires otherwise, the words comprise, comprises and comprising will be understood to imply the inclusion of a stated integer or group of integers but not the exclusion of any other integer or group of integers.

In order that the invention may be readily understood and put into practical effect, particular preferred embodiments will now be described by way of the following non-limiting examples.

### Step 1

Biological Sample Collection

A biological sample comprising nucleic acids, for example total RNA and mRNA, is collected. The biological sample may include cells of the immune system at various stages of development, differentiation and activity. The biological sample in most instances would be whole blood collected from a vein of a performance animal. However, the biological sample may include a fluid and/or tissue, for example sputum, urine, tissue biopsies, bronchial or nasal lavages, joint fluid, peritoneal fluid or thoracic fluid which, in part, comprises cells of the immune system that have infiltrated such tissues or fluids. Cells present in blood which comprise mRNA may include mature, immature and developing neutrophils, lymphocytes, monocytes, reticulocytes, basophils, eosinophils, macrophages. All of these cell types also appear in tissues of non-blood origin at various times in various conditions.

Methods described herein may include use of the above mentioned cell types. The biological sample is collected and prepared using various methods. For example, an easy method of collecting cells of the blood is by venipuncture. The biological sample may be collected from a performance animal, for example, a horse with suspected laminitis, a human athlete or camel with osteochondrosis, or a greyhound with subclinical cystitis.

Blood sample

Ten ml of blood is drawn slowly (to prevent hemolysis) from the vein of an animal Ougular vein in a horse and camel, veins on the forearm/limb of humans and dogs) into a 1:16 volume of 4% sodium citrate to prevent clotting and the sample is mixed and then placed on ice. The sample is centrifuged at 3000 RPM at 4° C. for 15 minutes and white blood cells (WBC) (commonly called the “buffy coat”) are removed from the interface between plasma and red blood cells (RBC) into a separate tube using a pipefte. The WBCs are then treated with at least 20 volumes of 0.8% ammonium chloride solution to lyse any contaminating RBC and re-centrifuged at 3000 RPM at 4° C. for 5 minutes. The pelletted WBCs are then washed in 0.9% sodium chloride, re-centrifuged, and kept on ice. The cell pellet is then used directly in RNA extraction.

Non-blood Biological Fluid Sample

A fluid sample, for example, sputum, urine, bronchial or nasal lavages, joint fluid, peritoneal fluid or thoracic fluid, is centrifuged at 3000 RPM at 4° C. for 20 minutes to collect cells. Samples comprising large amounts of mucous are treated with a mucolytic agent such as dithiothretol prior to centrifugation. A cell pellet is then washed in 0.9% sodium chloride, re-centrifuged and the cell pellet is used directly in RNA extraction.

Tissue Biopsy

A tissue biopsy is frozen in dry ice or liquid nitrogen and crushed to powder using a mortar and pestle. The frozen tissue is then used directly in RNA extraction.

### Step 2

RNA Isolation and Preparation

RNA Isolation

Total RNA and/or mRNA is isolated from a biological sample. Use of isolated mRNA rather than total RNA may provide results with less background and improved signal.

RNA is commonly isolated by skilled persons in the art, and examples of some methods for isolating mRNA are described below.

Commercially available kits, for example, Qiagen RNA and Direct RNA extraction kits, and RNA extraction kits produced by Invitrogen (formerly Life Technologies) and Amersham Pharmacia Biotech herein incorporated by reference, may be used by following the manufacturer's instructions. Key elements of these mRNA extraction protocols include use of an appropriate amount of sample, protection of the sample from RNAse contamination, elution of the sample from a column at 70° C. and quantitation and quality checking in an agarose 0.7% gel and using an OD 260/280 ratio. About 0.2 gm (wet weight) of pelleted white blood cells or tissue is required for each mRNA extraction which will yield about 1-2 μg of mRNA. Disposable gloves should be worn throughout the procedure, with frequent changes. Both the column and solution used for elution should be at 70° C.

RNA quantification and assessment of RNA size and quality include standard gel electrophoresis methods of running a small quantity of an RNA sample on an agarose gel with known standards, staining the gel with for example ethidium bromide to detect the sample and standards and comparing relative intensities and size of standard RNA and sample RNAs, comparison of the intensities of the ribosomal RNA bands. Alternatively, or in addition, RNA concentration in a solution may be determined by measuring absorbance at 260/280 nm in a spectrophotometer relative to known standards and calculated using known formulas.

cDNA Synthesis and Labelling

RNA prepared as described above may be synthesised to cDNA and labelled resulting in a labelled probe using kits provided by suppliers such as Amersham Pharmacia Biotech, Invitrogen, Stratagene or NEN, herein incorporated by reference. For example, a typical reaction may comprise: template RNA, an oligo-dT primer and/or gene-specific primers, reverse transcriptase enzyme, deoxyribonucleic triphosphates (dNTP), a suitable buffer, and a label incorporated into at least one of the dNTPs. Such a reaction when combined with a method of amplifying the resultant cDNA is referred to as RT-PCR (reverse transcriptase-polymerase chain reaction). A specific example is provided below, but it should be noted that other methods of incorporation of label into DNA can be used and that such methods are under constant review and improvement, for example some methods include the incorporation of amino-allyl dUTP and subsequent coupling of N-hydroxysuccinate activated dye to increase the specific labelling of the DNA.

To anneal primer(s) to template RNA, mix 2 μg of mRNA or 50-100 μg total RNA from respective test sample (Cy3) and reference sample (Cy5) in separate tubes with 4 μg of a regular or anchored oligo-dT primer or gene-specific primers in a total volume of 15 μl (using purified water to make up the volume). (Regular oligo dT is 5′-TTT TTT TTT TTT TTT TTT TTT, anchored oligo dT is 5′-TTT TTT TTT TTT ITT TTT TTV N-3′), (where V=A, C or G; and N=A, C, G or T). Heat mixture to 65 ° C. for 10 min and cool on ice. Add 15.0 μl of reaction mixture to respective Cy3and Cy5reactions.

The reaction mixture comprises of the following: 6.0 ul of 5× first-strand buffer, 3.0 μl of 0.1M DTT, 0.6 ul of unlabeled dNTPs, 3.0 ul of Cy3or Cy5dUTP (1 mM, Amersham), 2.0 ul of Superscript II (Reverse transcriptase 200 U/μL, Life Technologies) made to 15 μl with pure water. Unlabelled dNTPs are sourced from a stock solution consisting of 25 mM dATP, 25 mM dCTP, 25 mM dGTP, 10 mM dTTP. 5× first-strand buffer consists of 250 mM Tris-HCL (pH 8.3), 375 mM KCl, 15 mM MgCl₂). The mixture is incubated at 42° C. for 1 hr. Add an additional 1 μl of reverse transcriptase to each sample. Incubate for an additional 0.5-1 hrs. Degrade the RNA and stop the reaction by adding 15 μl of 0.1N NaOH, 2 mM EDTA and incubate at 65-70° C. for 10 min. If starting with total RNA, degrade the RNA for 30 min instead of 10 min. Neutralize the reaction by adding 15 μl of 0.1N HCl. Add 380 μl of TE (10 mM Tris, 1 mM EDTA) to a Microcon YM-30 column (Millipore).

Next add 60 μl of Cy5probe and 60 μl of Cy3probe to the same microcon. Centrifuge the column for 7-8 min. at 14,000×g. Remove flow-through and add 450 μl TE and centrifuge for 7-8 min. at 14,000×g (washing step). Remove flow-through and add 450 μl 1×TE, 20 μg of species-specific Cot1 DNA (20 μg/ul, Life Technologies for human—Cot1 DNA is genomic DNA that has been denatured and re-annealed such that the concentration of the DNA and the time of re-annealing multiplied equals 1. Methods for making Cot1 DNA are common in the art), 20 μg polyA RNA (10 μg/ul, Sigma, #P9403) and 20 μg tRNA (10 μg/ul, Life Technologies, #15401-011). Centrifuge 7-10 min. at 14,000×g. The probe needs to be concentrated such that with the addition of other solutions required for hybridisation the volume is not excessive, or is suitable for use with a desired slide and cover slip size. Invert the microcon into a clean tube and centrifuge briefly at 14,000 RPM to recover the probe.

A nucleic acid may be labelled with one or more labelling moieties for detection of hybridised labelled nucleic acid (ie. probe) and target nucleic acid complexes. Labelling moieties may include compositions that can be detected by spectroscopic, photochemical, biochemical, immunochemical, optical or chemical means. Labelling moieties may include radioisotopes, such as ³²P, ³²P or ³⁵S, chemiluminescent compounds, labelled binding proteins, heavy metal atoms, spectroscopic markers, such , as fluorescent markers and dyes, magnetic labels, linked enzymes, and the like. Preferred fluorescent markers include Cy3 and Cy5, for example available from Amersham Pharmacia Biotech (as decribed above).

cRNA Synthesis and Labelling

The Affymetrix system uses RNA as substrate and generates biotin labelled CRNA through a series of reactions detailed in a protocol available from their website (affymetrix.com), incorporated herein by reference. The cRNA is fragmented prior to application onto the array.

### Step 3

Arrays

One feature of the invention is an array comprising nucleic acids representing expressed genes from cells found in blood of a performance animal, for example a horse, human, camel or dog. The nucleic acids may be of any length, for example a polynucleotide or oligonucleotide as defined herein.

Each nucleic acid occupies a known location on an array. A nucleic acid target sample probe is hybridised with the array of nucleic acids and an amount or relative abundance of target nucleic acid hybridised to each probe in the array is determined.

High-density arrays are useful for monitoring gene expression and presence of allelic markers which may be associated with disease. Fabrication and use of high density arrays in monitoring gene expression have been previously described, for example in WO 97/10365, WO 92/10588 and U.S. Pat. No. 5,677,195, all incorporated herein by reference. In some embodiments, high-density oligonucleotide arrays are synthesised using methods such as the Very Large Scale Immobilised Polymer Synthesis (VLSIPS) described in U.S. Pat. No. 5,445,934, incorporated herein by reference.

Arrays for human are commercially available from companies such as Incyte, Research Genetics, and Affymetrix. Lion Bioscience recently announced forthcoming release of a dog microarray and have a clone collection of dog cDNAs. These arrays typically comprise between 2,000 and 10,000 genes and are species specific. None are available for the horse or camel. Some of these genes are in multiple copies on the array and have not been fully annotated or given a true gene identity. Additionally, it is not known whether DNA on the array, when hybridised to a test sample, specifically binds to a single gene. This latter instance results from splice variants of RNA transcripts in tissues such that one gene may encode multiple transcripts.

Human and dog arrays (when available) can be used in methods described herein. However, these arrays are currently non-specific and include genes that are not expressed in blood cells of animals, and/or do not contain genes important in controlling the function of blood cells, and/or contain regions of genes that are not specific to blood cells.

Clones containing specific genes are available and can be purchased for human (mouse and dog) for use on arrays (for example from the IMAGE consortium or Lion Bioscience). However, it is not possible to obtain specific clones for use on a blood-specific array without prior knowledge of what genes are expressed in blood cells. The IMAGE consortium also does not guarantee that the gene of interest is contained in the clone purchased.

Array Construction

Because of difficulties, problems and a likelihood of wasting financial resources to obtain a blood-specific DNA array, a method is provided herein which provides rapid and cost effective generation of species and tissue-specific DNA arrays for assessing nucleic acid expression in a sample. FIG. 3 shows steps for constructing an array in one embodiment.

Target Nucleic Acid Preparation

Biological samples are collected as described above. Samples comprising cells expressing as many genes of interest in relation to condition(s) of a performance animal are collected. For example, a sample comprising a mixture of n [symbol] ated blood cells from performance animals with conditions such as, osteochondrosis, laminitis, tendon soreness, bursitis, abcesses, inflammation, allergy, viral infection, parasite infection, asthma, etc.

Approximately 5 μg of mRNA is isolated from the biological sample (typically 1 gm wet weight) using mRNA isolation kits or the protocol described above. Concurrently, 5 μg of mRNA is isolated from umbilical cord blood, and/or early stage foetus. Cells and tissues contained within these sources would express genes that may not be expressed in the cells extracted from blood in the above example. Isolation of cytoplasmic mRNA from cells is preferred. This step involves rupturing the cells with a solution comprising detergent and/or chaotropic agent and salt such that cell nuclei and the nuclear membrane remain intact. The cell nuclei are pelleted by centrifugation and the supernatant is used for mRNA extraction. Protocols for this procedure are available as part of mRNA isolation kits (eg available by Qiagen). These mRNAs may be used to construct CDNA libraries. Kits for the construction of cDNA libraries are available from companies including Stratagene and Invitrogen (eg Uni-ZAP XR cDNA synthesis library construction kit #200450). The library preferably should be constructed such that the orientation of the CDNA in the vector is known, that the mRNA is primed using oligo dT, the vector is capable of receiving a nucleic acid insert up to 10 kb and that purification of DNA suitable for DNA sequencing is possible and easy. By following the manufacturer's instructions and paying particular attention to the quality of mRNA used and the size fractionation of cDNA (greater than 0.7 kb), a quality library containing enough viruses (>1×10⁶) with insert sizes >0.7 kb can be generated.

Plasmids generated from such a library can be DNA sequenced using protocols that are well established in the art and are available, for example, from Applied Biosystems. Briefly, a mix of 0.5 μg of plasmid DNA, 3.2 pmol of a primer that hybridises to the vector DNA (eg M13-21, or M13 reverse primer), thermostable DNA polymerase, dNTP and labelled dNTP is subjected to a routine PCR procedure to generate fragments of DNA that can be separated by gel electrophoresis and using machinery such as that available from Applied Biosystems (eg a 3700 DNA sequencer). Generated DNA sequence data (chromatogram) is assessed and quality scores and binning of similar sequences is done using a computer program package such as Phred/Phrap/Consed. The raw DNA sequence data can then be loaded into a database where comments (annotation) on the sequence can be made, such as quality score, bin, length of poly A sequence (should there be one), BLAST search results, highest homology in Genbank, clone identity, other entries in Genbank.

Subjective factors influencing whether a nucleic acid should be used on an array include quality and confidence of the DNA sequence, a Genbank homology score with identified nucleic acids, evidence of a poly-A tail (indicative of a translated transcript), uniqueness of the 3′ sequence data (compared to both Genbank and an in-house database of clone sequences).

Nucleic acid primers can be selected using a program such as Primer 3 available via the Internet (www-genome.wi.mit.edu/cgi-bin/primer/primer3). The selected primers may be used for amplifying a nucleic acid, for example by PCR, or directly applied to an array. Uniqueness of a nucleic acid can be tested by performing additional BLAST searches on Genbank and an in-house database. Primers are preferably designed such that melting temperatures are similar, and amplification products are of a similar nucleic acid length. Primers for PCR are generally between 18 and 25 nucleotide bases long. Primers for direct use on a microarray or device are preferably between 50 and 80 nucleotide bases long. Both the amplification product and the single primer should hybridise to DNA that uniquely identifies a gene transcript. Specific programs using various formulas are available for calculating the melting temperature of various lengths of DNA (eg Primer 3). Alternatively, selected DNA sequences can be provided to Affymetrix for production of a proprietary and custom array. The sequences generated in-house are provided to Affymetrix in Fasta format along with details of which parts of the sequence to be used for the generation of a probe set (11 probes, each 25 nucleotide bases long) for each gene represented on the array.

Nucleotide sequences may be compared with an existing database, for example Genbank, to determine a previously provided name, tissue expression, timing of expression, biochemical pathway, cluster membership, and possible function or cellular role of an expressed nucleic acid. In addition, a nucleic acid fragment may be used as a probe to isolate a full-length nucleic acid which may encode a gene which is associated with a particular disease or condition. Further, identified nucleic acids may be used to isolate homologues thereof, inclusive of orthologues from other species. An identified nucleic acid may also be cloned into a suitable expression vector to produce an expressed polypeptide in vitro, which may be used, for example as an antigen in generating antibodies and for use on protein arrays. The antibodies may be used for developing specific diagnostic assays or therapies, for three-dimensional protein structure such as X-ray crystallographic studies, or for therapeutic development.

An array may comprise any number of different nucleic acids, but typically comprises greater than about 100, preferably greater than about 1,000, more preferably greater than about 5,000 different nucleic acids. An array may comprise more than 1,000,000 different nucleic acids. Each nucleic acid is preferably represented more than once for scanning internal comparison and control. Preferably, the nucleic acids are provided in small quantities and are gene-specific and/or species-specific usually between 50 and 600 nucleotides long, arranged on a solid support.

The Affymetrix system uses 11 probes per gene, each of 25 nucleotides, that are built onto the array using a photolithographic method (U.S. Pat. Nos. 6,309,831; 6,168,948; 5,856,174; 5,599,695; 5,831,070; 6,153,743; 6,239,273; 6,271,957; 6,329,143; 6,310,189 and 6,346,413). The nucleic acids may be dotted onto the solid support or bound to microspheres, or in solution. A typical array may have a surface area of less than 1 cm², for example a microarray.

A nucleic acid can be attached to a solid support via chemical bonding. Furthermore, the nucleic acid does not have to be directly bound to the solid support, but rather can be bound to the solid support through a linker group. The linker groups may be of sufficient length to provide exposure to the attached nucleic acid. Linker groups may include ethylene glycol oligomers, diamines, diacids and the like. Reactive groups on the solid support surface may react with one of the terminal portions of the linker to bind the linker to the solid support. Another terminal portion of the linker is then functionalised for binding the nucleic acid. A solid support may be any suitable rigid or semi-rigid support, including charged nylon or nitrocellulose, chemically treated glass slides available from companies such as NEN, Corning, S&S, arrays available through Affymetrix, membranes, filters, chips, slides, wafers, fibers, magnetic or nonmagnetic beads, gels, tubing, plates, polymers, microparticles and capillaries. The solid support can have a variety of surface forms, such as wells, trenches, pins, channels and pores, to which the nucleic acids are bound. Preferably, the solid support is optically transparent.

The array may be constructed using an “arraying machine” manufactured by companies for example Molecular Dynamics, Genetic Microsystems, Hitachi, Biorobotics, Amersham, Corning. Alternatively, the array may be manufactured according to specific instructions provided by the user to Affymetrix. Source materials for this machine include microtitre plates comprising nucleic acids representative of unique genes, or sequence information. An array element may comprise, for example, plasmid DNA comprising nucleic acids specific for a gene sequence, an amplified product using gene-specific or non-specific primers and template DNA or RNA, or a synthesised specific oligonucleotide or polynucleotide. Array elements may be purified, for example, using Sephacryl-400 (Amersham Pharmacia Biotech, Piscataway, N.J.), Qiagen PCR cleanup columns, or high performance liquid chromatography (for oligonucleotides).

Purified array elements may be applied to a coated glass substrate using a procedure described in U.S. Pat. No. 5,807,522, incorporated herein by reference. By other example, DNA for use on Corning amino-silane coated slides (CMT-GAPS™) is re-suspended in 3×SSC to a concentration of 0.15-0.5 μg/μl and then used directly in an arraying machine in 96 or 384-well plates.

An example for preparing an array element is provided by the manganese superoxide dismutase gene. A clone comprising a nucleic acid insert is prepared and isolated as described above. The clone is sequenced to identify the nucleotide sequence. A BLAST search using the identified nucleotide sequence is performed to determine homology-of the cloned nucleic acid with nucleic acids in a database, for example GenBank. Identification of nucleotide sequence homology with superoxide dismutase genes stored in the database provides a level of confidence that the clone comprises at least in part a gene for superoxide dismutase for the horse. Unique primers can be designed to amplify a nucleic acid using PCR and the clone DNA, or genomic DNA from the same species as a template. Purified amplification product can be directly attached to an array and thereby act as a target for a complementary labelled nucleic acid probe in the test and reference samples. Alternatively, a unique sequence can be determined and an oliognucleotide manufactured and purified for direct use on an array, or the sequence information supplied directly to Affymetrix for the construction of a custom array.

The array may comprise negative and positive control samples (preferably as duplicates or triplicates) such as nucleic acids from species different from a sample being tested (negative controls) and various nucleic acids (representative of RNAs and both ends of RNA molecules) that are found in all tissues as a constant and known quantity (positive controls). These controls are identified and used by the array reader to provide data on true signal (ie. Specific hybridisation between probe and target) and noise (ie. Non-specific hybridisation between probe and target) and average intensity from multiple reads of several different locations for each nucleic acid attached to the array.

A test sample and a reference sample may be simultaneously assayed on the array. The reference sample may comprise mRNA from multiple sources, such that most, preferably all of the nucleic acids on the array are represented in the test sample, and can be used by the array reader as a non-zero standard and for comparison with an average of the read-outs from the test sample. A relative intensity for each gene on the array can be calculated.

The relative abundance of expression of each gene in a sample can also be calculated using controls within the array, such as certain genes expressed in a tissue at a constant level under all conditions.

Alternatively, using the Affymetrix system, an absolute level of expression is calculated based on the difference between the perfect match and mismatch hybridisation for each of the 11 probes for each gene. Using such a process a gene is scored as present or absent and an absolute measure of intensity is given along with a p value.

The interpreted array may highlight only a few genes that are substantially different in expression between a test and reference sample. Alternatively, the overall pattern of expression may provide a “fingerprint” to characterise the way in which the original cells have responded to a particular condition of a performance animal. For example, the gene for superoxide dismutase may be the only gene up-regulated in a particular condition, especially in conditions of inflammation, or a large number of genes may be up- and down- regulated in various conditions. It is this fingerprint, rather than specific knowledge of gene sequence or function that can be used as a marker for various conditions. It would be expected that fingerprints be useful across species barriers to include performance animals such as humans, horse, dog and camel.

The arrangement of nucleic acids on the array may be periodically changed and these arrays are then assigned a particular batch code which corresponds to a specific array comprising a specific nucleic acid arrangement. The ability to change the arrangement of nucleic acids on the array and knowledge of the exact arrangement may prevent other people from generating a database using the arrays produced by the present invention. Using a batch code also enables tracking of manufacturers of the arrays in regards to the number of arrays produced. The batch code further enables validation of a user of the communication network or “internet” diagnostic method and system. Batch code an also identify a particular type of array used, should more disease-specific arrays be designed and manufactured.

An example of how an array may be prepared and analysed is described in Eisen and Brown (Methods in Enzymology, 1999, 303 179) and in U.S. Pat. No. 6,114,114, herein incorporated by reference. Chapter 22 of Ausubel et al. supra also describes methods and apparatus for use with arrays and is herein incorporated by reference.

Control samples may be respectively labelled in parallel with a test and reference sample. Quantitation controls within a sample may be used to assure that amplification and labelling procedures do not change a true distribution of nucleic acid probes in a sample. For this purpose, a sample may include or be “spiked” with a known amount of a control nucleic acid which specifically hybridises with a control target nucleic acid. After hybridisation and processing, a hybridisation signal obtained should reflect accurately amounts of control nucleic acid added to the sample. For such purposes, a microarray may have internal controls, for example a nucleic acid encoding a common gene expressed by the performance animal with known expression levels and a nucleic acid encoding a gene from another species that is known not to hybridise to the test or reference sample. To improve sensitivity and specificity of the assay, blocking agents such as Cot DNA from the tested species may also be used.

### Step 4

Hybridising Sample Nucleic Acid Probes with an Array

Nucleic acid probes may be prepared as described above from a biological sample from a performance animal that has been assessed concurrently by physical inspection and/or blood tests or other method. Nucleic acid targets from a statistically relevant number of normal animals previously hybridised to arrays, and a reference range for each of the genes on the array is calculated and used as a normal reference range (for example a 95% population median). Results from a test sample from a test animal can be compared with the same genes as the normal reference to determine if the test sample falls within the normal reference range. Further, nucleic acid targets may also be prepared from biological samples from apparently normal animals, animals with overt disease, various progressive stages of disease, hitherto undiagnosed or unclassified conditions or stages of such conditions, animals treated with known amounts of drugs (legal or otherwise), animals suspected of being treated with drugs (legal or otherwise), animals under specific exercise regimes for the sake of performance, animals subjected to (intentional or not) various nutritional states and/or environmental conditions. Databases of information from the use of such samples and arrays are created such that test samples can be compared. The database will then contain specific patterns of gene expression for particular conditions.

Prior to hybridisation, a nucleic acid probe may be fragmented. Fragmentation may improve hybridisation by minimising secondary structure and/or cross-hybridisation with another nucleic acid probe in a sample or a nucleic acid comprising non-complementary sequence. Fragmentation can be performed t mechanical or chemical means common in the art.

A labelled nucleic acid target may hybridise with a complementary nucleic acid probe located on an array. Incubation conditions may be adjusted, for example incubation time, temperature and ionic strength of buffer, so that hybridisation occurs with precise complementary matches (high stringency conditions) or with various degrees of less complementarity (low or medium stringency conditions). High stringency conditions may be used to reduce background or non-specific binding. Specific hybridisation solutions and hybridisation apparatus are available commercially by, for example, Stratagene, Clontech, Geneworks.

Affymetrix have detailed a standard procedure for the hybridisation of probes with an array (as describe at their website, affymetrix.com, incorporated herein by reference), however, a typical method entails the following:

Adjust probe volume (prepared as above) to a value indicated in the “Probe & TE” column below according to the size of the cover slip to be used and then add the appropriate volume of 20×SSC and 10% SDS.

| Cover Slip | Total Hyb | Probe & TE | | |
|---|---|---|---|---|
| Size (mm) | Volume (μl) | (μl) | 20 × SSC (μl) | 10% SDS (μl) |
| 22 × 22 | 15 | 12 | 2.55 | 0.45 |
| 22 × 40 | 25 | 20 | 4.25 | 0.75 |
| 22 × 60 | 35 | 28 | 5.95 | 1.05 |

20 × SSC is 3.0 M NaCl, 300 mM NaCitrate (pH 7.0).

Denature the probe by heating it for 2 min at 100° C., and centrifuge at 14,000 RPM for 15-20 min. Place the entire probe volume on the array under the appropriately sized glass cover slip. Hybridize at 65 ° C. (temperatures may vary when using different hybridisation solutions) for 14 to 18 hours in a custom slide chamber (for example a Corning CMT hybridisation chamber #2551).

Washing the Array

After hybridisation, the array is washed to remove non-specific probe and dye hybridisation. Wash solutions generally comprise salt and detergent in water and are commercially available. The wash solutions are applied to the array at a predetermined temperature and can be performed in a commercially available apparatus. Stringency conditions of the wash solution may vary, for example from low to high stringency as herein described. Washing at higher stringency may reduce background or non-specific hybridisation. It is understood that standardisation of this step is required to produce maximum signal to noise ratio by varying the concentration of salt used, whether detergent is present (SDS), the temperature of the wash solution and the time spent in the wash solution.

A typical wash protocol consists of removing the slide from a slide chamber, removing the cover slip and placing the slide into 0.1% SSC (recipe provided above) and 0.1% SDS at room temperature for 5 minutes. Transfer the slide to 0.1% SSC for 5 minutes and repeat. Dry the slide using centrifugation or a stream of air. Equipment is available to enable the handling of more than one slide at a time (for example, slide racks).

### Step 5

Reading the Array

After removal of non-hybridised probe, a scanner or “array reader” is used to determine the levels and patterns of fluorescence from hybridised probes. The scanned images are examined to determine degree of hybridisation and the relative abundance of each nucleic acid on the array. A test sample signal corresponds with relative abundance of an RNA transcript, or gene expression, in a biological sample. Alternatively, an Affymetrix array is read and computer algorithms calculate the difference between hybridisation on perfect match and mismatch probes for each of the 11 probes sets for each gene. It then calculates a presence or absence, an absolute value for each gene and a p value for the absolute call.

Array readers are available commercially from companies such as Axon and Molecular Dynamics and Affymetrix. These machines typically use lasers, and may use lasers at different frequencies to scan the array and to differentiate, for example, between a test sample (labelled with one dye) and the control or reference sample (labelled with a different dye). For example, an array reader may generate spectral lines at 532 nm for excitation of Cy3, and 635 nm for excitation of Cy5.

A relative quantity of RNA may be calculated by the array reader and computer for respective nucleic acids on the array for respective samples based on an amount of dye detected, average of duplicate samples for respective genes and subtraction of background noise using controls. The reader is pre-programmed to perform such calculations (using proprietary software supplied with the array reader, such as MAS 5.0 for the Affymetrix system and Genepix for the Axon Instruments reader) and with information on the location of each nucleic acid on the array such that each nucleic acid is given a readout value. Controls or reference samples providing a readout for particular nucleic acids that falls within standard ranges ensures correct integrity of the array and hybridisation procedures. Programs typically generate digital data and format it for transmission

### Step 6

Querying and Transfer of Digital Data to a Central Database

Generated data is transmitted via a communications network to a remote central database. A user having access to the gene expression data enters information in relation to a test sample into a standard diagnostic form such that it can be digitalised. The information will include clinical appraisal and blood profile results. The format of such information is standard globally such that details on clinical conditions may be based on numerical input and each field of entry can be digitalised. For example, body temperature field could be number 0001, a recorded temperature within normal range would receive the number 0, 0.5° C. above what is considered to be the normal range for that species would receive a number 5, 1° C. above normal range would receive 10. Some examples of conditions that may be scored or rated in such a fashion are provided below.

a) Body temperature.

b) Integument: eyes, sores, abcesses, wounds, insects/parasites, allergy, infection.

C) Cardio/Respiratory: eyes, nasal discharge, rales, viral/bacterial infection, allergy, chronic obstructive pulmonary disease, cough/wheeze, crepitous sounds in the thorax, epistaxis, auscultation sounds, heart sounds, capillary refill, mucous membrane colour.

d) Gastrointestinal: diarrhoea, colic/stasis, parasites, appetite level, drenching time and dose.

e) Reproductive: stage of pregnancy, abortion, inflammation, discharges.

f) Musculoskeletal: lameness, laminitis, bone or shin soreness, muscle soreness or tying up, tendon or ligament affected, level of pain, X-ray data, scintigraphy data, CAT scan data, bursitis, bruising, cramping or “tying up”.

g) Blood test results: biochemistry, immunology, serology (viral, bacteriological, hormone levels), cell counts, cell morphology, pathologist interpretation.

h) Other diagnostic test results: X-ray, biopsy, histopathology, CAT scan, MRI, bacteriology, virology.

i) Other data: Season (date), location, male or female, vaccination history, body score (fitness and fat), fitness level.

Alternatively, the entire system could be based on the aforementioned SNOMED system with appropriate modifications to encompass descriptions of exercise physiology and the normal animal. Alternatively, the entire system could rely on text or categorical data that can be appraised and scored by software such as Omniviz. Whatever system is used, if would be appreciated that the aim is to adequately, systematically and in a standard manner describe the current condition of the animal to the best of currently available technologies and could include results from machinery such as X-ray, ultrasound, scintigraphy and blood analysis.

The user also ensures that array results (that may for example be automatically collected from a reader), array specifications, data mining specifications, level of interpretation required and the clinical information are entered and correspond to the same animal and the same sample. The form is transmitted electronically to a central database and recognised as an individual accession or request by the database. The central database recognises the user (using for example digital certificates), the user recognises the central database, the array batch code and gene array order are verified, and the user is allowed access (which may be automatic) and automatic processing of the request is performed if security and billing information are adequate. The processing involves specific mining of central data and specific user requested information is retrieved and resent automatically.

The above steps may be automated so that a user need not be present to perform the tasks. In an automated embodiment of the invention, gene expression data from an array reader may be transmitted via a communications network directly to a server which is connected to a central database. Additional information could be input by the user at a processor which is also linked to the array reader.

Automated Data Mining Using Sent Data (Heuristic Methods)

A central database interprets the array specifications (eg. nucleic acid order on a microarray), decodes the information transmitted, determines nucleic acid expression level in a biological sample and compares the expression level and patterns of expression with known standards or reference range. Various levels of database interpretation may be applied to the data transmitted, depending on the user requirements. Clusters of genes may be up-regulated or down-regulated in certain conditions and the database makes automated correlations to specific conditions by accessing various levels of database information.

Mining software such as Metamine (Silicon Genetics), ArraySCOUT (Lion Bioscience) can be used in this instance, and more advanced data mining technologies could be used to identify patterns and nearest neighbour information in data (such as products from AnVil Informatics Inc and OmniViz Inc). Further, software capable of taking rule-based instructions (such as that described by Pacific Knowledge Systems Sydney Australia in their “ripple down” technology) and having the ability to self learn (heuristics and neural network systems) such as that described in Khan et al. Nature Medicine 7 (6) 673, incorporated herein by reference, could be used at this stage to limit the level of human interaction in determining a diagnosis. In this latter example, an artificial neural network is used, and samples are divided into training and validation sets to create trained calibrated models. The calibrated models are then used to rank genes in diagnostic importance.

Levels of database may include:

Unique gene sequences (eg 3′ and 5′ EST sequence of genes)

Gene identity, homologous genes, tissue expression, keywords, function, cellular role, gene clusters, biochemical pathway, PubMed references

Primer sequences used to generate amplification products (eg two primer sequences used to uniquely amplify the gene for gamma interferon in a particular species)

Microarray construction and format (eg coded information on array manufacture batch and identification of genes and position on the array)

Blood profile and clinical data associated with particular conditions (eg standard clinical information and IDEXX-machine generated blood profile data)

Array data for normal and apparently normal status (eg 95% median range for normal animals)

Array data for inducible disease and disease models

Array data for various overt diseases (eg joint inflammation)

Array data for stages of various overt diseases (eg pre-clinical, clinical and recovery stages)

Array data for the influence of various classes of drugs, legal or otherwise, of known administration and dose, or unknown administration or dose (eg various steroids)

Array data for the response to known and various levels of drugs used as a therapy (eg various anti-inflammatory medication at specific doses for a specific condition)

Array data for the response to exercise and various training regimes

Array data for the response to nutrition and various feeding regimes

Array data for the response to the environment so as to possibly determine influence of during various seasons, or allergens or feed types.

Each successive level relies on at least one previous level of database to allow for interpretation. The database may be built over time and more intensive searching of the database may incur a greater cost. As the database grows, changes may be made to the above methodology to increase the sensitivity of the detection of variation in expression of condition-specific genes—this could include the use of condition-specific arrays or condition-specific primers. Condition-specific arrays can be manufactured by a company such as Affymetrix (under instructions) that would allow for increased sensitivity and specificity, much reduced size of arrays, decreased cost of production, and the ability to process multiple samples at once. The process of building the database is iterative, such that specific genes are correlated to specific conditions, and the detection of variations in these genes becomes more sensitive and specific through the use of various modifying processes through the procedure (eg. the use of gene-specific primers for the amplification and labelling of cDNA from RNA, and the selection of limited numbers of genes on a disease- or condition-specific array, detection of splice variants and single nucleotide polymorphisms).

### Step 7

Standardised Electronic Reporting

The database reports back electronically to a remote user, either automatically or with a level of human intervention. The electronic report may be converted to a printed document. The report provides details of an animal's condition that is determined by correlation of gene expression data with information stored in a remote database, and optionally expert analysis. Information sent might include:

Individual genes up-regulated or down-regulated (for example, with laminitis or joint capsule inflammation or bursitis, a report on the up-regulation of genes such as interleukin-3, manganese superoxide dismutase, Groα, metalloproteinase matix-metallo-elastase, ferritin light chain may have some correlation to tissue inflammation, and down-regulation of genes such as insulin-like growth factor and its receptor may be correlated to recovery from such a condition). The identity of these genes cannot be predicted to be associated to any condition unless the above described methodology is used and databases on relative expression of genes for particular conditions have been compiled. Therefore a screening test covering all genes may need to be performed first and a second, more specific test then applied.

The overall pattern of gene expression and any correlation to particular conditions. For example, animals in heavy training may have a gene “fingerprint” that is different to animals being spelled from training.

Individual pattern of gene expression (ie. the shape of the gene expression pattern over a time course or multiple samples taken over a period may change as an animal recovers from a condition)

Changes to a pattern of gene expression, gene expression profile or level for a single animal over a time period or for successive tests.

Clusters of genes up-regulated or down-regulated in a particular condition

Pathways of genes up-regulated or down-regulated in a particular condition

Correlations between genes up-regulated or down-regulated and known conditions, or stage of condition, or influence

Known therapies to ameliorate the condition or enhance desired effects

Specialist pathologist written interpretation

Relevant information of use to veterinarians, medical practitioners, owners, trainers and athletes

Collections of data on groups of animals under specific management regimes

Throughout the specification the aim has been to describe the preferred embodiments of the invention without limiting the invention to any one embodiment or specific collection of features. It would therefore be appreciated by those of skill in the art that, in light of the instant disclosure, various modifications and changes can be made in the particular embodiments exemplified without departing from the scope of the present invention. For example, the examples described herein may be used with performance animals other than horse, for example human, dog and camel.

All references, inclusive of patents, patent applications, scientific documents and computer programs, referred to in this specification are herein incorporated by reference in its entirety.

1. A method for assessing a performance status of a performance animal including the steps of:
a) determining for a plurality of expressed target nucleic acids in a sample obtained from the performance animal an abundance of the expressed target nucleic acids normalised to at least one reference nucleic acid;
b) providing the normalised abundance of the target nucleic acids as a digital sample signal;
c) transmitting the digital sample signal via a communications network to a remotely located diagnostic server, the diagnostic server having an associated processor and a database including digital information relating to a plurality of conditions, each condition being represented by abundances of selected one or more of the target nucleic acids; and,
d) causing the diagnostic server to process the digital sample signal by:
i) comparing the digital sample signal to the digital information to thereby identify any of the plurality of conditions of the performance animal;
ii) generating a report indicating whether the performance animal has one or more of the particular condition(s) thereby providing information on the performance status of the performance animal; and,
iii) returning the report to the user via the communications network.

2) The method of claim 1 wherein the sample comprises at least one immune cell type.

3) The method of claim 2 wherein the at least one immune cell type is a white blood cell.

4) The method of claim 1 wherein the normalised abundance of each target nucleic acid is an absolute abundance.

5) The method of claim 4 wherein the normalised abundance of each target nucleic acid is a relative abundance.

6) The method of claim 1 further including the step of determining from said sample or other sample obtained from the same performance animal as in step (a) one or more biological parameters and recording said parameters.

7) The method of claim 6 wherein said parameters are transmitted via a communications network to the same remotely located diagnostic server and associated processor and database of step (b).

8) The method of claim 7, the parameters being represented by the digital sample signal.

9) A method according to claim 1, the method including:
a) transferring the digital sample signal from a user to a transaction staging module via a first firewall; and,
b) transferring the digital sample signal from the transaction staging module to the database via a second firewall.

10) A method according to claim 8, the method of transferring the digital sample signal from the transaction staging module to the database via a second firewall including:
a) temporarily storing the digital sample signal in the transaction staging module;
b) causing a service module coupled to the database to generate a request;
c) causing the transaction staging module to transfer the digital sample signal to the service module via second firewall in response to the request.

11) The method of claim 1, wherein the digital information includes a number of gene expression profiles, each gene expression profile representing the abundance of target nucleic acids for a respective one of the conditions, the method including comparing the digital sample signal to the gene expression profiles to determine if the animal suffers from respective ones of the conditions.

12) The method of claim 1 wherein the communications network is selected from the group consisting of: the Internet, an intranet, an extranet, wireless means or dedicated link.

13) The method of claim 4 wherein the absolute abundance of each target nucleic acid is determined by the steps of:
i) detecting a first hybridised complex formed by at least one target nucleic acid and a perfect-complementary probe nucleic acid located on a solid support, thereby providing a digital perfect target signal;
ii) detecting a second hybridised complex formed by at least one target nucleic acid having a same nucleotide sequence as the target nucleic acid in step (i) and a mismatch-complementary probe nucleic acid comprising a mismatched nucleotide in a central location of the mismatch-complementary probe nucleic acid when compared with a corresponding perfect-complementary probe, wherein the mismatch-complementary probe nucleic acid is located on a solid support and hybridisation thereto provides a digital mismatch target signal; and
iii) comparing the digital perfect target signal of step (i) and the digital mismatch target signal of step (ii) to provide a digital sample signal of absolute abundance of the target nucleic acid.

14) The method of claim 13 wherein the respective hybridised complexes of step (i) and step (ii) are detectable by respectively labelling the target nucleic acids.

15) The method of claim 14 wherein the respectively labelled target nucleic acids are labelled with biotin, Cy3 or Cy5.

16) The method of claim 15 wherein the labelled target nucleic acid is cRNA.

17) The method of claim 13 wherein the solid support is an array.

18) The method of claim 17 wherein the array is a microarray.

19) The method of claim 5 wherein the relative abundance of each target nucleic acid is determined by the steps of:
(a) detecting a hybridised complex formed by at least one sample target nucleic acid and a complementary sample probe nucleic acid located on a solid support to provide a digital sample target signal;
(b) detecting a hybridised complex formed by at least one reference target nucleic acid comprising a nucleotide sequence different than the target nucleic acid of step (A), and a complementary reference probe nucleic acid located on a solid support to provide a digital reference target signal; and
(c) comparing the digital sample target signal of step (A) and the digital reference target signal of step (B) to provide a digital sample signal of relative abundance of the target nucleic acid.

20) The method of claim 19 wherein the respective complementary nucleic acids of step (A) and step (B) comprise a perfectly complementary or homologous nucleotide sequence.

21) The method of claim 19 wherein the respective hybridised complexes of step (A) and step (B) are detected by respectively labelling the sample target nucleic acid and the reference target nucleic acid.

22) The method of claim 21 wherein the respective sample target and the reference target nucleic acids are labelled with Cy3, Cy5 or biotin.

23) The method of claim 1 wherein the performance animal is a mammal.

24) The method of claim 23 wherein the mammal is selected from the group consisting of: human, horse, dog and camel.

25) The method of claim 1 wherein at least one of the conditions comprises an athletic ability and a condition that enhances, hinders, impedes or does not change an expected ability of said performance animal.

26) The method of claim 25 wherein at least one of the conditions comprises normal, apparently normal, pre-clinical disease, overt disease, progress and/or stage of disease, undiagnosed or unclassified conditions, presence of drugs, response to exercise, response to vaccines, therapies, nutritional states and response to environmental conditions.

27) The method of claim 26 wherein the disease comprises inflammation or involvement of the immune system; a condition affecting respiratory, musculoskeletal, urinary, gastrointestinal and adnexa, cardiovascular, reticuloendothelial, nervous, special senses, reproductive, and integument systems.

28) The method of claim 27 wherein the disease comprises laminitis, lameness, viral or bacterial disease, colic, gastritis, gastric ulcers, respiratory ailments, epistaxis, fractures, musculoskeletal damage or disorders and joint disease in the horse.

29) A method according to claim 1, the method including comparing of the relative abundances of selected expressed target nucleic acids with a reference range of corresponding selected target nucleic acids, a successful comparison indicating the presence of a respective condition.

30) A method according to claim 1 at least one of the nucleic acids being used in the identification of more than one condition.

31) A diagnostic system for assessing a performance status of a performance animal, the diagnostic system comprising:
i) an array comprising a plurality of probe nucleic acids immobilised to a surface, wherein the respective probe nucleic acids comprise nucleotide sequences hybridisable to a plurality of target nucleic acids;
ii) an array reader that detects hybridised complexes formed respectively by the target nucleic acids and the probe nucleic acids, whereby the array reader generates a digital sample signal of the respective detected hybridised complexes;
iii) a remotely located database storing digital information in relation to a plurality of conditions of performance animals, each condition being represented by abundances of selected one or more of the target nucleic acids, and clinical and blood profile data;
iv) a diagnostic server that:
(1) receives the digital sample signal via a communications network;
(2) compares the digital sample signal with the digital information in the database to identify any of the plurality of conditions;
(3) generates a report indicating whether the performance animal has one or more of the particular condition(s) thereby providing information on the performance status of the performance animal; and,
(4) transfers the report to the user via the communications network.

32) The system of claim 31 wherein the probe nucleic acid is selected from the group consisting of: a perfect-complementary nucleic acid comprising a nucleotide sequence perfectly complementary to the target nucleic acid, a mismatch-complementary nucleic acid comprising a mismatched nucleotide in a central location of the nucleic acid when compared with a corresponding perfect-complementary nucleic acid, and a reference nucleic acid comprising a nucleotide sequence that is different than the target nucleic acid and hybridisable to a complementary reference target nucleic acid.

33) The system of claim 32 further comprising a means to display the report.

34) The system of claim 33, the diagnostic server being adapted to compare the relative abundances of selected expressed target nucleic acids with a reference range of corresponding selected target nucleic acids, a successful comparison indicating the presence of a respective condition.

35) The system of claim 34, wherein the digital information includes a number of gene expression profiles, each gene expression profile representing the abundance of target nucleic acids for a respective one of the conditions, the diagnostic server being adapted to compare the digital sample signal to the gene expression profiles to determine if the animal suffers from respective ones of the conditions.

36) The system of claim 31, at least one of the nucleic acids being used in the identification of more than one condition.

37) A system according to claim 31, the system including:
a) a first firewall for transferring a request from a user to a transaction staging module, the request including the digital sample signal; and,
b) a second firewall for transferring the digital sample signal from the transaction staging module to the database.

38) A system according to claim 37, the digital sample signal being stored in the transaction staging module, the system including a service module adapted to:
a) generate a signal request;
b) transfer the signal request to transaction staging module, the transaction staging module being adapted to transfer the digital sample signal to the service module via second firewall in response to the request.

39) A method for assessing a performance status of a performance animal including the steps of:
a) determining for a plurality of expressed target nucleic acids in a sample obtained from the performance animal an abundance of the expressed target nucleic acids normalised to at least one reference nucleic acid;
b) providing the normalised abundance of the target nucleic acids as a digital sample signal to a diagnostic server, the diagnostic server having an associated processor and a database including a number of gene expression profiles, each gene expression profile representing abundances of selected one or more of the target nucleic acids for a respective condition;
c) causing the diagnostic server to process the digital sample signal by:
i) comparing the digital sample signal to the gene expression profiles; and,
ii) determining whether the performance animal has one or more of the particular condition(s) thereby providing information on the performance status of the performance animal.
